SEQUENCE LISTING (3 sequences)

SEQ ID NO: 1 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 2 - protein, 232 aa, Homo sapiens
MVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEE

SEQ ID NO: 3 - protein, 238 aa, Homo sapiens
MVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEEHHHHHH

(19) United States
(12) Patent Application Publication
(10) Pub. No.: US 2024/0368253 A1
(43) Pub. Date: Nov. 7, 2024

(54) HUMAN VITRONECTIN FRAGMENTS AND USES THEREOF

(71) Applicant: BIO-TECHNE CORPORATION, Minneapolis, MN (US)

(72) Inventors: Kevin C. Flynn, Hastings, MN (US); Marnelle Andersen, Woodbury, MN (US); Jun Li, Eden Prairie, MN (US); Amy Isaacson, Crystal, MN (US); Bryan J. Jones, Minneapolis, MN (US)

(21) Appl. No.: 18/686,506

(22) PCT Filed: Aug. 31, 2022
(86) PCT No.: PCT/US2022/042228

§ 371 Date: Feb. 26, 2024

Related U.S. Application Data
(60) Provisional application No. 63/239,456, filed on Sep. 1, 2021.

(51) Int. Cl. C07K 14/78; C12N 5/074; C12N 5/0775

(52) U.S. Cl. CPC C07K 14/78; C12N 5/0662; C12N 5/0696; C07K 2319/21; C12N 2533/52

(57) ABSTRACT

The invention relates to human vitronectin fragments with improved manufacturability in E. coli, improved function in stem cell culture applications. and improved stability in comparison to wild-type vitronectin or previously characterized vitronectin fragments. The present invention relates to the uses thereof as a substrate for cell culture and in regenerative medicine.

### CONTINUING APPLICATION DATA

This application claims the benefit of U.S. Provisional Application Ser. No. 63/239,456, filed Sep. 1, 2021, which is incorporated by reference herein.

### SEQUENCE LISTING

This application contains a Sequence Listing electronically submitted to the United States Patent and Trademark Office via Patent Center as an XML file entitled “0541-000017WO01” having a size of 5 kilobytes and created on Aug. 23, 2022. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### BACKGROUND

Regenerative medicine is a growing field of medicine with the goal of repairing, regrowing, or replacing injured or diseased cells, tissues, and organs. Both mesenchymal stem cells (MSCs) and pluripotent stem cells (PSCs) (including embryonic and induced pluripotent stem cells (iPSCs)) are currently in clinical trials for various indications in regenerative medicine. An essential aspect of regenerative medicine is the cultivation and expansion of stem cells and their derivatives ex vivo for use as therapeutics for tissue replacement strategies (Blau and Daley, 2019, N Engl J Med; 380(18):1748-1760). A key step in generating these cells is their safe and robust ex vivo cultivation. This entails optimization of cell culture media and substrates that are safe and dependable (from a supply standpoint), while maintaining robust functionality. In regard to safety, standards have been outlined in the US Pharmacopeia (USP) publication USP<1043> (USP43-NF38-7381, USP-NF<1043> “Ancillary Materials for Cell, Gene, and Tissue Engineered Products,” Phamacopeial Forum; 43:7381), which details a tiered system for risk classification of raw materials used as ancillary materials in cell and gene therapy, including regenerative medicine (USP43-NF38). The use of animal containing reagents is considered high risk (Tier 4) while animal-free materials generated under GMP conditions are lower risk (Tier 2). Achieving low-risk, animal-free substrates for the cultivation of stem cells is thus an important goal for advancing broader applications in regenerative medicine.

Conventional cell culture techniques for MSCs and for PSCs use either plasma purified matrices such as fibronectin (Veevers-Lowe et al., 2011, Journal of Cell Science; 124(Pt 8):1288-300; and Somaiah et al., 2015, PLoS One; 10(12):e0145068) or complex tumor derived matrices such as MATRIGEL™ or CULTREX BME™ (Xu et al., 2001, Nat Biotechnol; 19(10):971-4). More recently, matrices with more homogenous compositions made with recombinant manufacturing protocols (including laminin, fibronectin, and vitronectin) have been used to cultivate stem cells (Miyazaki et al., 2012, Nat Commun; 3:1236; Miyazaki et al., 2013, Erratum in: Nat Commun; 4:1931; Braam et al., 2008, Stem Cells; 26(9):2257-65; and Kalaskar et al., 2013, J R Soc Interface; 10(83):20130139). These substrates can reduce variability and improve the definition of the cell culture environment. However, these proteins or protein fragments can be challenging to manufacture, expensive, and/or are made in animal cells.

There is a need in the field of regenerative medicine for easy to manufacture, robust, and truly animal-free matrix molecules.

### SUMMARY OF THE INVENTION

The present disclosure includes a vitronectin polypeptide fragment, wherein the vitronectin polypeptide fragment: comprises the deletion of at least 50 consecutive N-terminal amino acids relative to a full length vitronectin polypeptide having SEQ ID NO: 1; comprises the deletion of at least C-terminal amino acids 362 to 478 relative to the full length vitronectin polypeptide having SEQ ID NO: 1; comprises the arginine-glycine-aspartic acid (RGD) integrin binding domain of vitronectin; does not comprise the N-terminal somatomedin B (SmB) domain of vitronectin; does not comprise the C-terminal heparin binding domain and the Hp4 domain of vitronectin; and comprises about 95% sequence identity to the same fragment of the full length vitronectin polypeptide having SEQ ID NO: 1.

In some aspects, a vitronectin polypeptide fragment comprises the Hp1, Hp2, and/or Hp3 domains of vitronectin.

In some aspects, a vitronectin polypeptide fragment has a length of about 200 to about 350 amino acids.

In some aspects, a vitronectin polypeptide fragment comprises: the deletion of 61 consecutive N-terminal amino acids relative to a full length vitronectin polypeptide having SEQ ID NO: 1; and the deletion of C-terminal amino acids 293 to 478 relative to the full length vitronectin polypeptide having SEQ ID NO: 1.

In some aspects, a vitronectin polypeptide fragment comprises about 95% sequence identity to residues 62 to 292 of the full length vitronectin polypeptide having SEQ ID NO: 1.

In some aspects, a vitronectin polypeptide fragment comprises an amino acid substitution at a position equivalent to position 80 and/or position 148 of full length human vitronectin having SEQ ID NO: 1. In some aspects, the amino acid substitution comprises a D80Y substitution and/or a Q148E substitution.

In some aspects, a vitronectin polypeptide fragment comprises one or more amino acid substitutions at a position equivalent to position 63, position 67, and/or position 68 of full length human vitronectin having SEQ ID NO: 1. In some aspects, the one or more amino acid substitutions comprises a T63G substitution and/or a V67S substitution and/or a V67N substitution and/or a F68P substitution. In some aspects, the amino acid substitutions comprise a T63G substitution, a V67S substitution or V67N substitution, and a F68P substitution.

The present disclosure includes a vitronectin polypeptide fragment consisting of SEQ ID NO: 2.

The present disclosure includes a vitronectin polypeptide fragment consisting of SEQ ID NO: 2, having an amino acid substitution at position D80 and/or an amino acid substitution at position Q148 relative to full length vitronectin having SEQ ID NO: 1. In some aspects, the amino acid substitution at position D80 comprises a D80Y substitution and/or the amino acid substitution at position Q148 comprises a Q148E substitution. The present disclosure includes a vitronectin polypeptide fragment consisting of SEQ ID NO: 2, having an amino acid substitution at position T63 and/or an amino acid substitution at position V67 and/or F68 relative to full length vitronectin having SEQ ID NO: 1. In some aspects, the amino acid substitution at position T63 comprises a T63G substitution and/or the amino acid substitution at position V67 comprises a V67S or a V67N substitution. In some aspects, the amino acid substitution at position F68 comprises a F68P substitution.

In some aspects, a vitronectin polypeptide fragment as disclosed herein further comprises a C-terminal His-tag. In some aspects, comprising one, two, three, four, five, six, seven, eight, or more C-terminal His residues.

The present disclosure includes a vitronectin polypeptide fragment consisting of SEQ ID NO: 3.

In some aspects, a vitronectin polypeptide fragment as disclosed herein is animal-free.

In some aspects, a vitronectin polypeptide fragment as disclosed herein is conjugated to a microcarrier. In some aspects, the microcarrier comprises a hydrogel or polystyrene microsphere.

The present disclosure includes compositions of a vitronectin polypeptide fragment as disclosed herein or a vitronectin polypeptide fragment conjugate as disclosed herein. In some aspects, the composition is animal-free.

In some aspects, a vitronectin polypeptide fragment as disclosed herein, a vitronectin polypeptide fragment conjugate as disclosed herein, or a composition as disclosed herein is for use as a cell culture substrate.

The present disclosure includes a nucleotide sequence encoding a vitronectin polypeptide fragment as disclosed herein.

The present disclosure includes an expression vector comprising a nucleotide sequence as disclosed herein. In some aspects, the expression vector comprises an E. coli. expression vector.

The present disclosure includes a host cell comprising a comprising a nucleotide sequence as disclosed herein or an expression vector as disclosed herein. In some aspects, the host cell comprises E. coli.

The present disclosure includes a method of producing a vitronectin polypeptide fragment, the method comprising expressing a vitronectin polypeptide fragment from a nucleotide sequence as disclosed herein, an expression vector as disclosed herein, or a host cell as disclosed herein. In some aspects, the vitronectin fragment polypeptide produced is animal-free.

The present disclosure includes a cell culture method, the method comprising culturing cells on a substrate comprising a vitronectin polypeptide fragment as disclosed herein, a vitronectin polypeptide fragment conjugate as disclosed herein, or a composition as disclosed herein. In some aspects, the cells comprise stem cells. In some aspects, the cells are cultured under animal-free conditions. In some aspects, the cell culture is ex vivo.

As used herein, “isolated” refers to material removed from its original environment (e.g., the natural environment if it is naturally occurring), and thus is altered “by the hand of man” from its natural state.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

The recitations of numerical ranges herein by endpoints include all numbers subsumed within that range (e.g., 1 to 5 includes 1, 1.5, 2, 2.75, 3, 3.80, 4, 5, etc.).

Herein, “up to” a number (for example, up to 50) includes the number (for example, 50).

The term “in the range” or “within a range” (and similar statements) includes the endpoints of the stated range.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

In several places throughout the application, guidance is provided through lists of examples, which examples can be used in various combinations. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

All headings throughout are for the convenience of the reader and should not be used to limit the meaning of the text that follows the heading, unless so specified.

### BRIEF DESCRIPTION OF THE FIGURES

FIG. 1 shows the linear structure of vitronectin with functional domains. The full vitronectin polypeptide chain (top) and variants generated in this study are shown. The integrin binding RGD domain is labeled at residues 64-66. The functional domains are as follows: somatomedin B (SmB), hemoepoxin (Hp), and heparin binding (Hb). There is a disulfide bond between cysteines at position 293 and 430.

FIGS. 2A and 2B show a comparison of human iPSC cells cultured on the different VTN fragments at 1 μg/ml, 5 μg/ml, and 10 μg/ml. Here full length recombinant VTN, made in NSO cells, is included as a control. As shown in FIG. 2A, iPSC cells grown on VTN62-120 and VTN62-155 result in small round colonies while VTN62-292 supports the growth of large colonies. Representative brightfield images of the iPSCs are shown. In FIG. 2B, quantification of the fold expansion of the iPSCs after 4 days in culture is shown in the bar graphs. Note that of the variants, only the hVTN62-292 fragment can support iPSC cell expansion at the low concentration and that the VTN-N variant shows reduced expansion in comparison.

FIGS. 3A and 3B show a comparison of human MSC cells cultured on the different VTN fragments at 10 μg/ml and 5 μg/ml and 1 μg/ml. As shown in FIG. 3A, while the morphology of the MSCs are similar on all VTN variants, the numbers are reduced on VTN62-120 and VTN62-155. Representative brightfield images of the MSCs are shown. Quantification of the fold expansion of the MSCs after 2 passages in culture is shown in FIG. 3B. Note that of the bacterially expressed variants, the hVTN62-292 fragment displays superior MSC cell expansion at low concentrations (1 μg/ml).

FIGS. 4A to 4D show the long-term maintenance of iPSCs in a pluripotent state when cultured on hVTN62-292. In FIG. 4A, iPSCs are maintained on the VTN62-292 variant with an animal-free iPSC media maintain normal morphology and healthy expansion over 5 passages. The bar graph shows the average number of total viable cells per well after 4 days of growth from 100,000 cells starting population. After long-term cultures, iPSCs maintain markers for pluripotency as indicated by flow cytometry in FIG. 4B. The dark gray histogram shows the antibody signal and the light gray histogram show the isotype control staining. The iPSCs maintain high levels of the stemness markers Oct3, Sox2, and SSEA-4. Conversely, the iPSCs have low levels of SSEA-1, a differentiation marker for human iPSCs. The percent positive cells are indicated in the upper right of the flow graphs. Gates on the flow graphs were set by the isotype control. As shown in FIG. 4C, iPSCs cultured on VTN62-292 maintain their capacity to differentiate into ectoderm cells. The images show the transcription factor Otx2 to visualize cells differentiated into ectoderm and DAPI to visualize total cell numbers. The graph indicates the increase in the percent of cells expressing Otx2 after ectoderm differentiation compared to undifferentiated iPSCs. As shown in FIG. 4D, iPSCs cultured on VTN62-292 maintain their capacity to differentiate into definitive endoderm. The images show the staining of the transcription factor Sox 17 to visualize endoderm differentiated cells and DAPI to visualize total cell numbers. The graph indicates the increase in the percent of cells expressing Sox 17 after endoderm differentiation compared to undifferentiated iPSCs.

FIGS. 5A to 5C show that following the long-term maintenance of iPSCs on hVTN62-292, they retain the capacity to differentiate into Neural Progenitor Cells (NPCs) and Forebrain neurons in a completely animal-free workflow. FIG. 5A is a diagram showing the experimental workflow. iPSCs are maintained on the VTN62-292 variant with an animal-free iPSC media. Neural induction occurs with the inhibitor SB43219 and a N2-GMP supplement. Neural progenitor cells appear after 7-10 days after neural induction. After over 28 days, 2 passages, and conversion into a neuronal media with an animal-free N21 supplement, forebrain neurons emerge. As shown in FIG. 5B, IBJ6 iPSC neurons were cultured under conditions to maintain stemness (day 0, d0), differentiated into intermediate neural progenitor cells (NPCs) after 10 days (d10), or differentiated into terminal forebrain neurons after 32 days (d32). The images show naïve iPSCs express high levels of Oct3/4, which is greatly diminished after 10 days of NPC differentiation (left panels). After NPC differentiation, there is an increase in Pax6 expression, which is not observed in the naïve iPSCs (middle panels). After 32 days of neural differentiation, there are a prevalence of neurons with long beta-3-tubulin positive neurites, which are absent in naïve iPSCs (right panels). The smaller images show DAPI staining to visualize the cells via the nuclei. The bar graphs of FIG. 5C indicate that the majority of iPSCs (>80%) are positive for Oct3/4, but this stemness marker is lost in the differentiated neurons. Conversely, iPSCs do not express beta-III-tubulin, while over 60% of differentiated cells express high levels of this neuronal marker.

FIGS. 6A and 6B show that mutations conceived to improve integrin binding do not result in improved function for iPSC cell culture while those designed based on inter-species consensus sequences show similar (Q148E and D80Y) iPSC cell expansion. FIG. 6A is brightfield images of iPSCs cultured on 1 μg/ml VTN variants showing that iPSCs colonies grow well on VTN,D80Y and VTN,Q148E, but are diminutive on VTN,T63G, VTN,T63G, V67N,F68P, and VTN,TG63, V67S. The bar graphs of FIG. 6B show total cell numbers of two separate iPSC cells (BYS110 and IBJ6) on the VTN variants coated at two different concentrations. Note that for both the BYS110 and IBJ6 cell lines, the D80Y shows similar viable cell counts compared to the other mutants, especially at 1 μg/ml (light gray box). The triple mutant VTN, T63G, V67N,F68P did not support adhesion or iPSC cell expansion and the double mutant VTN,T63G, V67S,F68P had attenuated growth compared to the VTN62-292 variant (FIGS. 6A and 6B).

FIGS. 7A to 7C show the binding affinity of the VTN variants to alpha VBeta5 (αVβ5) integrin heterodimers in assays. FIG. 7A shown representative graphs of ELISA titration curves. Y-axis shows Optical Density (O.D.) measurements from HRP secondary signals. The X-Axis shows the titration of the protein. The table shown in FIG. 7B summarizes ED50 (median effective dose) values for different VTN variants in one experiment. The bar graph of FIG. 7C shows the ED50 average of 5 ELISA experiments normalized to VTN-FL. In these assays, hVTN62-292 displays a modest increased, though similar binding affinity to αVβ5 as full-length VTN (VTN-FL). These both display similar affinity to αVβ5 as the VTN-N variant (VTN-CTS) which is within 7% of VTN-FL. Surprisingly, a VTN mutant postulated to improve integrin binding, VTN62-292 T63G, V67N, F68P greatly diminished αVβ5 binding affinity, with an over 7-fold increase in the ED50 value. However, the D80Y mutation displayed enhanced αVβ5 affinity with a reduction of over 44% in the average ED50 value. Error bars=standard error.

FIG. 8 shows the binding of cells to the various vitronectin variants, as determined in cell adhesion assays. In this assay, iPSCs were allowed to adhere to low concentrations of the VTN variants (0.56 ng/ml) for 1 hour, washed and assessed for adhesion. There is nearly a complete loss of cell adhesion to the VTN62-292T63G, V67N,F68P mutant. In comparison to the VTN-N variant, there is an increase to the VTN62-292 variant alone, with no further increase in adhesion with the Q148E nor the D80Y mutant.

FIG. 9 shows VTN62-292 thermostability. The data is based on cell expansion relative to full length Vitronectin (VTN-FL). When incubated at 37 degrees Celsius for 4 days before plating cells, there is a notable decrease in the ability VTN-N to support iPSC expansion. However, VTN62-292 displays no obvious reduction in cell growth relative to VTN-FL. The D80Y and Q148E mutations do not seem to confer any advantage for stability as predicted. Error bars=standard deviation.

FIGS. 10A-10D show VTN62-292 supports the pluripotency of iPSCs as shown via tri-lineage differentiations analyzed via flow cytometry. FIGS. 10A-10C present the results of flow cytometry experiments showing increased expression Brachyury, HNF4α, and Pax6 for endoderm (FIG. 10A), ectoderm (FIG. 10B), and mesoderm (FIG. 10C), as shown by the significant increase in mean fluorescent intensity for these markers. In addition, the stemness marker Nanog significantly decreases following a protocol to induce mesoderm differentiation

(FIG. 10D).

FIGS. 11A and 11B show VTN62-292 supports the long-term culture of the iPSCs with genomic stability as indicated by karyotype analysis. After long-term culture of iPSCs on VTN62-292 for over 2 months and 13 passages, cells maintain a normal karyotype, indicating genomic stability, an important feature for the propagation of stem cells for regenerative medicine.

FIGS. 12A to 12D show the reprogramming of peripheral blood monocytes (PBMCs) into induced pluripotent stem cells using hVTN62-292 as the substrate. This is significant in that previous work failed to show this with other VTN variants (Ye and Wang, 2018, Cell Physiol Biochem; 50(4):1318-1331). FIG. 12A shows the formation of iPSC colonies at different times following transduction with the Sendai Cytotune 2.0 reprogramming kit (Thermo-Fischer). After cloning individual colonies, nascent iPSCs derived on hVTN62-292 exhibit the morphological features of iPSCs with tightly packed cells in colonies with smooth borders. These iPSCs display the characteristic markers for iPSCs including Nanog, Oct3/4 and E-Cadherin expression (FIG. 12B). The analysis of Oct3 and Sox2 by flow cytometry also showed the expression of stemness markers while the absence of SSEA1 expression indicating an undifferentiated state of the iPSCs (FIG. 12C). In addition, the iPSCs derived from a single clone have the differentiation capacity to form mesoderm cells as indicated by the increased expression of Brachyury (FIG. 12D).

FIGS. 13A and 13B shows VTN62-292 and various mutants display altered affinity to integrin receptors. The binding affinity of the VTN62-292 variant and mutations flanking the RGD motif to the major RGD-binding integrins heterodimers including alpha VBeta5 (αVβ5), alphaVBeta1 (αVβ1), alphaVBeta3 (αVβ3) and alpha5Beta5 (α5β1) was assayed via enzyme-linked immunosorbent assays (ELISAs). FIG. 13A shows representative graphs of ELISA titration curves. Y-axis shows Optical Density (O.D.) measurements from HRP secondary signals. The X-Axis shows the titration of the protein. The tables shown in FIG. 13B summarizes ED50 (median effective dose) values for different VTN variants from representative experiments.

FIG. 14 shows VTN62-292 and various mutants thereof display altered affinity to integrin receptors as shown by relative ELISA binding affinities. Theses bar graphs show the ED50 average of ≥2 ELISA experiments normalized to Fibronectin (FN) (for α5β1, αVβ1, and αVβ3) or VTN-FL (αVβ5). In these assays, hVTN62-292 displays a modest increase, though similar binding affinity to αVβ5 as full-length VTN (VTN-FL). However, hVTN62-292 shows improved binding affinity to αVβ3, αVβ1, and α5β1. The VTN-N variant (VTN-CTS) shows a similar trend, but typically with lower affinity for all 4 integrin heterodimers. These experiments confirmed that the VTN mutants postulated to improve integrin binding, VTN62-292 T63G, V67N, F68P greatly diminished αVβ5 binding affinity, with an increase in the ED50 value. The VTN62-292 T63G, V67S, F68P mutant also showed reduced αVβ5 binding affinity. The triple mutants did show increased affinity to the two major fibronectin receptors, αVβ3 and α5β1, as well as αVβ1. This is important in that here are mutants that are similar to fibronectin in their integrin binding capacity. The graphs for αVβ3, αVβ1 and α5β1 are relative to fibronectin binding and the graph for αVβ5 is relative to full length vitronectin. Error bars=standard deviation.

FIGS. 15A and 15B show the effects of VTN mutants on MSC growth in culture. The data presented is from triplicate experiments of three concentrations of VTN. The cell growth was monitored by analyzing the confluence of the MCS cell culture after 72 hours. FIG. 15A shows brightfield images of MSCs on FN and the different VTN62-292 mutants. All three concentrations show that the VTN62-292, T63G, V67N,F68P and VTN62-292, T63G, V67S,F68P mutants support enhanced MSC growth (FIG. 15B). This is in sharp contrast to the growth of iPSCs on the triple mutants which is attenuated (VTN62-292, T63G,V67S,F68P) or completely abrogated (VTN62-292,T63G,V67N,F68P) (as shown in FIGS. 6A and 6B).

FIGS. 16A and 16B show the cell expansion of the IBJ6 iPSC cell line in the undifferentiated state and following differentiation into MSCs on FN, VTN62-292, and VTN62-292, T63G, V67N,F68P. This experiment shows that the same cell line at different states of stemness can switch their substratum preference. In the undifferentiated state, iBJ6 iPSCs only expand significantly on VTN62-292 as seen in brightfield images and quantified by percent confluence (FIG. 16A). Note there is essentially no growth on VTN62-292, T63G, V67N,F68P. Following differentiation to induced MSCs (iMSCs), the same cell line show increased growth on FN but the best growth on VTN62-292,T63G,V67N,F68P (FIG. 16B). This is likely due to the increased expression of αVβ3 following MSC differentiation and/or increased binding through α5β1 integrins.

### DETAILED DESCRIPTION OF ILLUSTRATIVE EMBODIMENTS

The present disclosure provides vitronectin polypeptide fragments with improved properties for production and use as a substrate for cell culture, including for the ex vivo culture of stem cells in animal-free conditions.

Vitronectin (VTN or VN) is a glycoprotein of the hemopexin family which is abundantly found in serum and the extracellular matrix that is involved in cell adhesion, migration, cell spreading, and extracellular anchoring. The full length vitronectin polypeptide is 478 amino acid residues. With removal of the signal sequence, the mature vitronectin protein is a 54 kDa glycoprotein of 459 amino acid residues.

The amino acid sequence of the full length human vitronectin (NCBI Accession number NM_000638.4) is shown below:

(SEQ ID NO: 1)
MAPLRPLLILALLAWVALADQESCKGRCTEGFNVDKKCQCDELCSYYQS
CCTDYTAECKPQVTRGDVFTMPEDEYTVYDDGEEKNNATVHEQVGGPSL
TSDLQAQSKGNPEQTPVLKPEEEAPAPEVGASKPEGIDSRPETLHPGRP
QPPAEEELCSGKPFDAFTDLKNGSLFAFRGQYCYELDEKAVRPGYPKLI
RDVWGIEGPIDAAFTRINCQGKTYLFKGSQYWRFEDGVLDPDYPRNISD
GFDGIPDNVDAALALPAHSYSGRERVYFFKGKQYWEYQFQHQPSQEECE
GSSLSAVFEHFAMMQRDSWEDIFELLFWGRTSAGTRQPQFISRDWHGVP
GQVDAAMAGRIYISGMAPRPSLAKKQRFRHRNRKGYRSQRGHSRGRNQN
SRRPSRATWLSLFSSEESNLGANNYDDYRMDWLVPATCEPIQSVFFFSG
DKYYRVNLRTRRVDTVDPPYPRSIAQYWLGCPAPGHL.

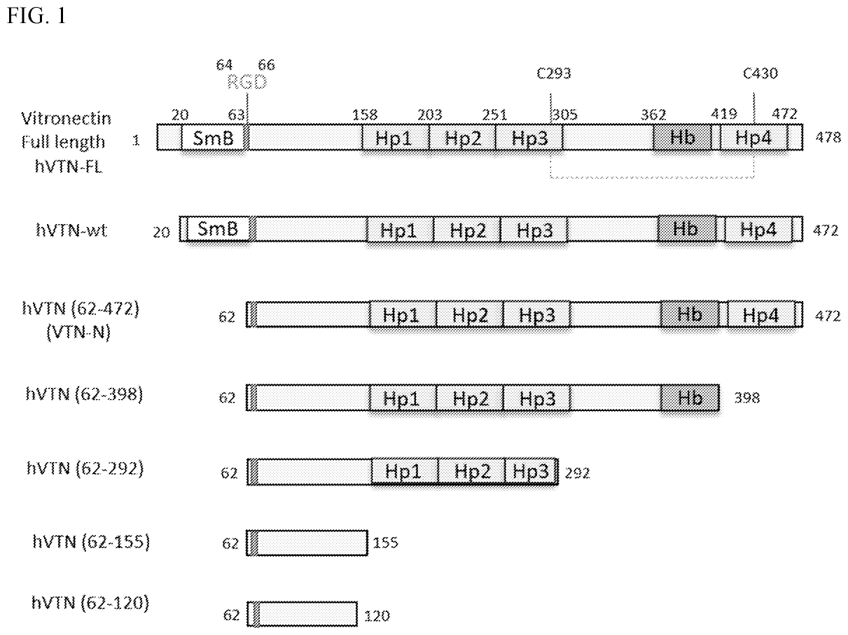

The secondary structure of full length vitronectin is illustrated in FIG. 1, showing the following domains:
A signal peptide from amino acids residues 1 to 19.
An N-terminal Somatomedin B (SMB) domain at amino acid residues 20 to 63. The SMB domain is a compact disulfide knot, with 4 disulfide bonds within about 35 residues.
An Arginine-Glycine-Aspartic Acid (RGD) integrin binding motif at amino acid residues 64 to 66.
Three central domains with hemopexin homology. These hemopexin-like domains are Hp1 from amino acids residues 158-202, Hp2 from amino acid residues 203 to 250, and Hp3 from amino acid residues 251-305.
A heparin binding domain (Hb) that includes amino acid residues 365 to 395;
A C-terminal domain with hemopexin homology (Hp4) at amino acid residues 419 to 472.

Vitronectin polypeptide fragments of the present disclosure include vitronectin polypeptide fragments with the deletion of one or more of the signal peptide, the N-terminal Somatomedin B (SMB) domain, the heparin binding domain (Hb), and/or the C-terminal Hp4 domain. In some aspects, a vitronectin polypeptide fragment of the present disclosure includes a vitronectin polypeptide fragment in which the signal peptide, the N-terminal Somatomedin B (SMB) domain, the heparin binding domain (Hb), and the C-terminal Hp4 domain have been deleted.

In some aspects, a vitronectin polypeptide fragment of the present disclosure includes the arginine-glycine-aspartic acid (RGD) integrin binding domain of vitronectin at amino acid residues 64 to 66.

In some aspects, a vitronectin polypeptide fragment of the present disclosure may include any one, any two, or all three of the Hp1, Hp2, and Hp3 domains of vitronectin.

In some aspects, a vitronectin polypeptide fragment of the present disclosure includes a vitronectin polypeptide fragment that includes the arginine-glycine-aspartic acid (RGD) integrin binding domain of vitronectin at amino acid residues 64 to 66 and includes any one, any two, or all three of the Hp1, Hp2, and Hp3 domains of vitronectin and in which the signal peptide from amino acids, the N-terminal Somatomedin B (SMB) domain, the heparin binding domain (Hb), and the C-terminal Hp4 domain have been deleted.

A vitronectin polypeptide fragment of the present disclosure has a length of less than that of full length vitronectin (478 amino acids) or mature vitronectin (459 amino acids). For example, a vitronectin polypeptide fragment of the present disclosure may have a length of about 200 amino acids to about 350 amino acids, about 200 amino acids to about 300 amino acids, about 200 amino acids to about 250 amino acids, about 250 amino acids to about 350 amino acids, or about 300 amino acids to about 350 amino acids. A vitronectin polypeptide fragment of the present disclosure may have a length of about 200, about 210, about 220, about 230, about 240, about 250, about 260, about 270, about 280, about 290, about 300, about 310, about 320, about 330, about 340, or about 350 amino acids, or any range thereof. A vitronectin polypeptide fragment of the present disclosure may have a length of about 230, about 231, about 232, about 232, about 234, bout 235, about 236, about 237, about 239, about 240 amino acids, or any range thereof.

A vitronectin polypeptide fragment of the present disclosure may include the deletion of the 61 consecutive N-terminal amino acids and the deletion of C-terminal amino acids 293 to 478 relative to a full length vitronectin polypeptide having SEQ ID NO: 1. In some aspects, such a vitronectin polypeptide fragment is the fragment of amino acids 62 to 292 of human vitronectin (SEQ ID NO: 1), also referred to herein as hVTN (62-292), VTN62-292, or h Vitronectin: 62-292, having the amino acid sequence:

(SEQ ID NO: 2)
MVTRGDVFTM PEDEYTVYDD GEEKNNATVH EQVGGPSLTS
DLQAQSKGNP EQTPVLKPEE EAPAPEVGAS KPEGIDSRPE
TLHPGRPQPP AEEELCSGKP FDAFTDLKNG SLFAFRGQYC
YELDEKAVRP GYPKLIRDVW GIEGPIDAAF TRINCQGKTY
LFKGSQYWRF EDGVLDPDYP RNISDGFDGI PDNVDAALAL
PAHSYSGRER VYFFKGKQYW EYQFQHQPSQ EE

A vitronectin polypeptide fragment of the present disclosure includes vitronectin polypeptide fragments at least about 75%, at least about 80%, at least about 85%, at least about 90%, at least about 95%, at least about 96%, at least about 97%, at least about 98%, or at least about 99% identical to the vitronectin polypeptide fragment of SEQ ID NO: 2. As used herein “sequence identity” between two polypeptides is determined by comparing the amino acid sequence of one polypeptide to the sequence of a second polypeptide. When discussed herein, whether any particular polypeptide is at least 40 percent (%), at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identical to another polypeptide may be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman (1981) Advances in Applied Mathematics 2:482-489, to find the best segment of homology between two sequences. When using BESTFIT or any other sequence alignment program to determine whether a particular sequence is, for example, 95% identical to a reference sequence according to the present disclosure, the parameters are set such that the percentage of identity is calculated over the full-length of the reference polypeptide sequence and that gaps in homology of up to 5% of the total number of amino acids in the reference sequence are allowed.

A vitronectin polypeptide fragment of the present disclosure includes vitronectin polypeptide fragments having one, two, three, four, five, six, seven, eight, nine, ten, or more amino acid substitutions to SEQ ID NO: 2. Such amino acid substitutions include, but are not limited to, any of those shown in Table 1.

Such amino acid substitutions may be in positions flanking the RGD integrin binding site, in the region corresponding to amino acids 62-70 of SEQ ID NO: 1. For example, such substitutions may be at:
position 63 of full length human vitronectin having SEQ ID NO: 1 (position 3 of SEQ ID NO: 2), including but not limited to a T63G amino acid substitution;
position 67 of full length human vitronectin having SEQ ID NO: 1 (position 7 of SEQ ID NO: 2), including but not limited to a V67S amino acid substitution;
position 68 of full length human vitronectin having SEQ ID NO: 1 (position 8 of SEQ ID NO: 2), including but not limited to a F68P amino acid substitution;
positions 63 and 67 of full length human vitronectin having SEQ ID NO: 1, including but not limited to a T63G or a V67S amino acid substitution; and/or
positions 63, 67, and 68 of full length human vitronectin having SEQ ID NO: 1, a T63G, including but not limited to a V67N, and a F68P amino acid substitution.

Such an amino acid substitution may include a substitution at position 80 of full length human vitronectin having SEQ ID NO: 1 (position 20 of SEQ ID NO: 2), including but not limited to a D80Y amino acid substitution.

Such an amino acid substitution may include a substitution at position 143 of full length human vitronectin having SEQ ID NO: 1 (position 83 of SEQ ID NO: 2), including but not limited to a H143D amino acid substitution.

Such an amino acid substitution may include a substitution at position 148 of full length human vitronectin having SEQ ID NO: 1 (position 88 of SEQ ID NO: 2), including but not limited to a Q148E amino acid substitution.

Such an amino acid substitution may include a substitution at position 149 of full length human vitronectin having SEQ ID NO: 1 (position 89 of SEQ ID NO: 2), including but not limited to a P149S amino acid substitution.

Such an amino acid substitution may include a substitution that replaces the cysteine at C293 of full length human vitronectin having SEQ ID NO: 1.

In some embodiments, a vitronectin polypeptide fragment of the present disclosure includes the fragment corresponding to amino acids 62 to 292 of human vitronectin (SEQ ID NO: 1) in which the aspartic acid (Asp, D) at position 80 has been replaced with a tyrosine (Tyr, Y) (the D at position 20 of SEQ ID NO: 2 has been replaced with a Y), referred to herein as VTN62-292,D80Y (also referred to herein as VTN62-292(D80Y), VTN62-292 D80Y, and VTN62-292D80Y).

In some embodiments, a vitronectin polypeptide fragment of the present disclosure includes the fragment corresponding to amino acids 62 to 292 of human vitronectin (SEQ ID NO: 1) in which the glutamine (Gln, Q) at position 148 has been replaced with a glutamic acid (Glu, E) (the Q at position 20 of SEQ ID NO: 2 has been replaced with an E), also referred to herein as VTN62-292 (Q148E).

In some embodiments, a vitronectin polypeptide fragment of the present disclosure may include the mutations VTN62-292, T63G, V67N,F68P and VTN62-292, T63G, V67S,F68P. The VTN fragments with these mutations may confer modifications in the binding to the RGD-binding integrins, namely alphaVBeta5 (αVβ5), alphaVBeta1 (αVβ1), alphaVBeta3 (αVβ3) and alpha5Beta5 (α5β1) in a manner similar to fibronectin binding to these integrins. As such and given the difficulty to express fibronectin (FN) in an animal-free system such as E.coli, the vitronectin polypeptide fragments and variants thereof disclosed herein provide a FN-like molecule that can be produced at scale in animal free systems. This is beneficial for additional applications in cell therapy and regenerative medicine that are improved via signaling through the FN receptors, αVβ3 and α5β1. Examples of such advantages included cardiomyocyte differentiation and osteoblast differentiation.

Also included in the present disclosure are vitronectin polypeptide fragments with additional heterologous amino acid residues not of vitronectin origin at the N-terminus and/or the C-terminus of the vitronectin polypeptide fragment. Such heterologous amino acid residues may, for example, encode an enzymatic activity, or other additional components, such as, for example, a linker, a detectable marker, or a fusion protein.

In some embodiments, the C-terminus of a vitronectin polypeptide fragment of the present disclosure may be conjugated to a detectable marker, such as for example, a poly-histidine tag or a streptavidin tag. Fluorescently conjugated antibodies directed against the poly-histidine tag or labeled biotin can be used to indirectly detect the vitronectin. These tags can provide quantifications related to protein amount and interactions with corresponding integrin receptors. In addition, these tags can be utilized in conjugations to hydrogel polymers (alginate, polyethylene glycol, etc.) via antibody conjugations. Alternatively, the vitronectin peptides can be directly conjugated via reactive groups such as free amine groups, carboxylic acid groups or sulfones to hydrogels for the formation of vitronectin-functionalized hydrogels. In some embodiments, the C-terminus may be modified with the addition of lysines, to increase side-chain reactive amino groups, or cysteines, to increase reactive thiols, for conjugation reactions.

In some embodiments, a vitronectin polypeptide fragment of the present disclosure includes the fragment corresponding to amino acids 62 to 292 of human vitronectin (SEQ ID NO: 1) with a C-terminus poly-histidine tag. The poly-histidine tag may be two, three, four, five, six, seven, eight, or more His residues, including, for example, the fragment corresponding to amino acids 62 to 292 of human vitronectin (SEQ ID NO: 1) with a C-terminus poly-histidine tag of six histidine residues, also referred to herein as Human Vitronectin Fragment 62-292 C-His or VTN62-292his, hVitronectin: 62-292/His6, having the following sequence:

(SEQ ID NO: 3)
MVTRGDVFTM PEDEYTVYDD GEEKNNATVH EQVGGPSLTS
DLQAQSKGNP EQTPVLKPEE EAPAPEVGAS KPEGIDSRPE
TLHPGRPQPP AEEELCSGKP FDAFTDLKNG SLFAFRGQYC
YELDEKAVRP GYPKLIRDVW GIEGPIDAAF TRINCQGKTY
LFKGSQYWRF EDGVLDPDYP RNISDGFDGI PDNVDAALAL
PAHSYSGRER VYFFKGKQYW EYQFQHQPSQ EEHHHHHH

A histidine tag serves many purposes. It provides for affinity purification to a nickel column, thereby isolating a vitronectin polypeptide fragment from other cellular material in a cell lysate. It can serve as an antigen for anti-His antibodies and used for the quantification of a vitronectin polypeptide fragment in immunoassays. And the His tag can be used to bind a vitronectin polypeptide fragment to a cell culture substrate.

A vitronectin polypeptide fragment of the present disclosure may posses one or more of the properties as described herein. The characterization of a vitronectin polypeptide fragment of the present disclosure may be by any of a variety of available methods, including, but not limited to, any of those described in the examples section included herewith.

For example, a vitronectin polypeptide fragment of the present disclosure may demonstrate improved yields in comparison to full length vitronectin or other fragments when expressed in host cells, such as for example, E. coli.

A vitronectin polypeptide fragment of the present disclosure may demonstrate improved solubility in comparison to full length vitronectin or other fragments when expressed in host cells, such as for example, E. coli.

A vitronectin polypeptide fragment of the present disclosure may demonstrate increased thermostability in comparison to full length vitronectin or other fragments.

A vitronectin polypeptide fragment of the present disclosure, including, but not limited to the mutations VTN62-292, T63G, V67N,F68P and VTN62-292, T63G, V67S,F68P, may demonstrate a decreased binding affinity to an RGD-binding integrin, for example, alpha VBeta5 (αVβ5), alphaVBeta1 (αVβ1), alphaVBeta3 (αVβ3) or alpha5Beta5 (α5β1). Decreased binding may be determined relative to full length vitronectin (SEQ ID NO:1) or to full length fibronectin.

A vitronectin polypeptide fragment of the present disclosure, including, but not limited to the mutations VTN62-292, T63G, V67N,F68P and VTN62-292, T63G, V67S,F68P, may demonstrate an increased binding affinity to an RGD-binding integrin, for example, alphaVBeta5 (αVβ5), alphaVBeta1 (αVβ1), alphaVBeta3 (αVβ3) or alpha5Beta5 (α5β1). Increased binding may be determined relative to full length vitronectin (SEQ ID NO:1) or to full length fibronectin.

A vitronectin polypeptide fragment of the present disclosure may serve as a suitable cell binding substrate for the adhesion/attachment of various cell types, including, but not limited to, human pluripotent stem cells (for example, embryonic stem cells and induced pluripotent stem cells) and mesenchymal stem cells, to cell culture surfaces.

A vitronectin polypeptide fragment of the present disclosure may serve as a cell culture matrix for two-dimensional cell culture (for example, the coating of tissue culture plastic), for conjugation to microcarriers (for example, hydrogel-based or polystyrene microspheres), or for culture in three dimensional matrices (for example, as a component in synthetic hydrogels).

A vitronectin polypeptide fragment of the present disclosure may support the undifferentiated proliferation and expansion of various cell types in culture, including, but not limited to, human pluripotent stem cells (for example, embryonic stem cells and induced pluripotent stem cells (iPSCs)) and mesenchymal stem cells (MSCs), in which the cells maintain their undifferentiated phenotype.

A vitronectin polypeptide fragment of the present disclosure may have an enhanced ability to support pluripotent stem cell viability, proliferation, pluripotency, cloning cell maintenance, differentiation, and/or induced pluripotent cell derivation in comparison to full length vitronectin or full length fibronectin.

A vitronectin polypeptide fragment of the present disclosure may support the differentiation of stem cells in differentiation protocols. For example, supporting the differentiation of human pluripotent stem cells (hPSCs) into mesendodermal and hepatic lineage cells or supporting the osteogenic differentiation of human mesenchymal stem cells (hMSCs).

A vitronectin polypeptide fragment of the present disclosure may be produced in animal-free conditions.

A vitronectin polypeptide fragment of the present disclosure may serve as a substrate for cell culture, including the ex vivo culture of stem cells, in animal-free conditions.

### Nucleic Acids

In another aspect, this disclosure describes an isolated polynucleotide molecule encoding a vitronectin polypeptide fragment as described herein. In some embodiments, the isolated polynucleotide molecule includes a nucleotide sequence that has at least 75%, at least 80%, at least 85%, at least 90%, at least 95%, at least 96%, at least 97%, at least 98%, or at least 99% sequence identity to nucleotide sequence encoding a vitronectin polypeptide fragment as described herein. In some embodiments, the isolated polynucleotide molecule includes a nucleotide sequence that has at least 75%, at least 80%, at least 85%, at least 90%, at least 95%, at least 96%, at least 97%, at least 98%, or at least 99% sequence identity to nucleotide sequence encoding a vitronectin polypeptide fragment of human vitronectin having SEQ ID NO: 1.

In another aspect, this disclosure describes recombinant vectors including an isolated polynucleotide of the present disclosure. The vector may be, for example, in the form of a plasmid, a viral particle, or a phage. The appropriate DNA sequence may be inserted into a vector by a variety of procedures. In general, the DNA sequence is inserted into an appropriate restriction endonuclease site(s) in a vector by procedures known in the art. Such procedures are deemed to be within the scope of those skilled in the art. Large numbers of suitable vectors and promoters are known to those of skill in the art and are commercially available. The following vectors are provided by way of example. Bacterial vectors include, for example, pQE70, pQE60, pQE-9, pBS, pD10, phagescript, psiX174, pbluescript SK, pbsks, pNH8A, pNH16a, pNH18A, pNH46A, ptrc99a, pKK223-3, pKK233-3, pDR540, and pRIT5. Eukaryotic vectors include, for example, pWLNEO, pSV2CAT, pOG44, pXT1, pSG, pSVK3, pBPV, pMSG, and pSVL.

In some aspects, a suitable vector is an expression vector for expression of a vitronectin polypeptide fragment in a prokaryotic host cell, a non-eukaryotic host cell, or a non-animal host cells (a host cell not of the Kingdom Animalia). Non-limiting examples include, for example, an expression vector for expression in prokaryotic host cells such as E. coli, fungal host cells such as Pichia pastoris, or plant host cells. However, any other plasmid or vector may be used.

In a further aspect, this disclosure also includes a host cell containing at least one of the above-described nucleotide sequences or vectors. The host cell may be a higher eukaryotic cell, such as a mammalian or insect cell, or a lower eukaryotic cell, such as a yeast cell. Or, the host cell may be a prokaryotic cell, such as a bacterial cell, a fungal cell, or a plant cell. In some embodiments, the host cell is E. coli. In some embodiments, the host cell is Pichia pastoris. Introduction of a vector construct into the host cell may be by any suitable techniques, such as, for example, calcium phosphate transfection, DEAE-Dextran mediated transfection, electroporation, or nucleofection. Vitronectin polypeptide fragments of the present disclosure may be expressed in mammalian cells, plant cells, yeast, bacteria, or other cells under the control of appropriate promoters. Examples of eukaryotic cells for such recombinant expression include HEK293, CHO, NSO, Vero, COS, HeLa, SF 21, cells, Pichia pastoris, Saccharomyces cerevisiae, and Oryza sativa cells. Prokaryotic expression systems include E. coli variants such as DH5 alpha, DE3, and ROSETTA® (Novagen) cell strains.

Cell-free translation systems may be employed to produce a vitronectin polypeptide fragment of the present disclosure, using RNAs derived from the DNA constructs of the present disclosure. Alternatively, vitronectin polypeptide fragments of the present disclosure may be chemically synthesized by procedures known in the art.

As shown in the examples included herewith the expression of vitronectin polypeptide fragments of the present disclosure in a host cell, such as E. coli, may demonstrate improved yields and improved solubility in comparison to the yield and solubility of full length vitronectin, or previously known fragments such as VTN62-398 and VTN62-478. The full-length natural version of human vitronectin it is difficult if not impossible to express in E. coli in large quantities, likely due to posttranslational processing of the protein. The same is true compared to larger fragments that include amino acids C293 and C430 which form a disulfide bond. When expressed in E. coli, fragment VTN62-398 demonstrates low expression and oxidation and the commercially available VTN fragment VTN62-478 (Cell Therapy Systems) accumulates in inclusion bodies and requires additional solubilization steps complicating large scale manufacturing. In contrast, vitronectin polypeptide fragments of the present disclosure, including, for example, the vitronectin polypeptide fragment of SEQ ID NO: 2 and SEQ ID NO: 3 and variants thereof, yield a soluble protein product when expressed in host cells such as E. coli.

The vitronectin polypeptide fragments of the present disclosure may be expressed or synthesized as described herein to obtain a preparation of the vitronectin polypeptide fragment that is animal-free, free from animal contamination. An animal-free product may, for example be expressed and isolated from a non-animal host cell, such as, for example, E. coli. An animal-free product may be made without the use of any animal containing raw materials in primary, secondary, or tertiary processes. The ex vivo cultivation of cells, including, but not limited to immune cells and stem cells, for therapeutic applications requires cultivation conditions that are safe and robust. Safety standards for the cell culture media and substrates used for such cultivation have been outlined in the US pharmacopeia (USP) publication USP<1043> which details a tiered system for risk classification of raw materials used as ancillary materials in cell and gene therapy, including regenerative medicine (USP43-NF38). The use of animal containing reagents is considered high risk (Tier 4) while animal-free materials generated under GMP conditions are lower risk (Tier 2). The vitronectin polypeptide fragments of the present disclosure provide such low-risk, animal-free products for the ex vivo cultivation of cells for therapeutic applications.

Also included in the present disclosure are compositions including one or more of the vitronectin polypeptide fragments described herein. Such compositions may be used in cell culture, including as substrates for cell adhesion. Such compositions may be animal-free. Such compositions may serve as a defined culture medium, a culture medium in which every constituent of the medium is fully disclosed and characterized.

A vitronectin polypeptide fragment of the present disclosure may be associated to or conjugated to a variety of substrates, including two-dimensional or three-dimensional cell culture substrates. A vitronectin polypeptide fragment of the present disclosure may be conjugated to a microcarrier, including, for example, a hydrogel or polystyrene microsphere. A hydrogel may be a dissolvable hydrogel. A hydrogel may include a composition of a plurality of alginic acid molecules and a plurality of branched polymer molecules in which the plurality of alginic acid molecules is conjugated to or blended with the branched polymer molecule to form a hydrogel as described in more detail in U.S. Pat. Nos. 9,927,334, 9,790,467, and 10,739,338, each of which is incorporated herein in its entirety.

The vitronectin polypeptide fragments described herein, conjugates thereof, and compositions thereof may be used in the culture of a variety of cell types, for use in applications from basic cell biology research to cell therapy and regenerative medicine workflows. Such vitronectin polypeptide fragments can serve as a cell attachment and/or growth substrate for various cell types.

Such culture may include, in addition to in vitro culture, the ex vivo of cells. As used herein, ex vivo means “outside of a living body.” With ex vivo culture, cells or tissue may be taken directly from a living organism and cultured under defined culture conditions. Such cell may then be returned to the organism after ex vivo culture.

The vitronectin polypeptide fragments described herein, conjugates thereof, and compositions thereof may be used in the culture of stem cells and cells derived from stem cells. including for example, adult stem cells, fetal stem cells, progenitor cells, peripheral hematopoietic stem cells, endothelial progenitor cells, amniotic stem cells, mesenchymal stem cells, adipose-derived stem cells, intestinal stem cells, skin stem cells, neural stem cells, or cancer stem cells. Stem cells may include pluripotent stem cells and multipotent stem cells (Salzig et al., 2016, Stem Cells Int; 2016:5246584; Braam et al., 2008, Stem Cells; 26(9):2257-65). Stem cell derived cells include but are not limited to mesoderm and cells derived therefrom such as cardiomyocytes, osteoblasts, chondrocytes, definitive endoderm, and cells derived therefrom, ectoderm and cells derived therefrom.

As used herein, the term “pluripotent stem cell” refers to a cell capable of differentiating into cells of all three germ layers. Examples of pluripotent cells include embryonic stem (ES) cells and induced pluripotent stem (iPS) cells. As used herein, “iPS cells” refer to pluripotent cells derived from somatic cells that display characteristics similar to higher potency cells, such as ES cells. Examples of multipotent stem cells include mesenchymal stem cells.

The vitronectin polypeptide fragments described herein, compositions thereof, and conjugates thereof may be used in the culture of cells for the engineering and/or expansion of cells for use in adaptive T cell therapy systems, such as for example, chimeric antigen receptor (CAR) T-cell immunotherapy (reviewed in, for example, in Feins et al., 2019, Am J Hematol; 94(S1):S3-S9; Mohanty et al., 2019, Oncol Rep; 42(6):2183-2195; and Huang et al., 2020, J Hematol Oncol, 13(1):86).

The invention is defined in the claims. However, below there is provided a non-exhaustive listing of non-limiting exemplary aspects. Any one or more of the features of these aspects may be combined with any one or more features of another example, embodiment, or aspect described herein. Exemplary Embodiments of the present invention include, but are not limited to, the following.

Embodiment 1. A vitronectin polypeptide fragment, wherein the vitronectin polypeptide fragment:
comprises the deletion of at least 50 consecutive N-terminal amino acids relative to a full length vitronectin polypeptide having SEQ ID NO: 1;
comprises the deletion of at least C-terminal amino acids 362 to 478 relative to the full length vitronectin polypeptide having SEQ ID NO: 1;
comprises the arginine-glycine-aspartic acid (RGD) integrin binding domain of vitronectin;
does not comprise the N-terminal somatomedin B (SmB) domain of vitronectin;
does not comprise the C-terminal heparin binding domain and the Hp4 domain of vitronectin; and
comprises about 95% sequence identity to the same fragment of the full length vitronectin polypeptide having SEQ ID NO: 1.

Embodiment 2. The vitronectin polypeptide fragment of Embodiment 1, wherein the vitronectin polypeptide fragment comprises the Hp1, Hp2, and/or Hp3 domains of vitronectin.

Embodiment 3. The vitronectin polypeptide fragment of Embodiment 1 or 2, wherein the vitronectin polypeptide fragment has a length of about 200 to about 350 amino acids

Embodiment 4. The vitronectin polypeptide fragment of any one of Embodiments 1 to 3, wherein the vitronectin polypeptide fragment comprises:
the deletion of 61 consecutive N-terminal amino acids relative to a full length vitronectin polypeptide having SEQ ID NO: 1; and
the deletion of C-terminal amino acids 293 to 478 relative to the full length vitronectin polypeptide having SEQ ID NO: 1.

Embodiment 5. The vitronectin polypeptide fragment of any one of Embodiments 1 to 4, wherein the vitronectin polypeptide fragment comprises about 95% sequence identity to residues 62 to 292 of the full length vitronectin polypeptide having SEQ ID NO: 1.

Embodiment 6. The vitronectin polypeptide fragment of any one of Embodiments 1 to 5, comprising an amino acid substitution at a position equivalent to position 80 and/or position 148 of full length human vitronectin having SEQ ID NO: 1.

Embodiment 7. The vitronectin polypeptide fragment of Embodiment 6, wherein the amino acid substitution comprises a D80Y substitution and/or a Q148E substitution.

Embodiment 8. A vitronectin polypeptide fragment consisting of SEQ ID NO: 2.

Embodiment 9. A vitronectin polypeptide fragment consisting of SEQ ID NO: 2, having an amino acid substitution at position D80 and/or an amino acid substitution at position Q148 relative to full length vitronectin having SEQ ID NO: 1.

Embodiment 10. The vitronectin polypeptide fragment of Embodiment 9, wherein the amino acid substitution at position D80 comprises a D80Y substitution and/or the amino acid substitution at position Q148 comprises a Q148E substitution.

Embodiment 11. The vitronectin polypeptide fragment of any one of Embodiments 1 to 10, comprising an amino acid substitution at a position equivalent to position 63, position 67, and/or position 68 of full length human vitronectin having SEQ ID NO: 1.

Embodiment 12. The vitronectin polypeptide fragment of Embodiment 11, wherein the amino acid substitution at position 63 comprises a T63G substitution, the amino acid substitution at position 67 comprises a V67S substitution or a V67N substitution, and/or the amino acid substitution at position 68 comprises a F68P substitution.

Embodiment 13. The vitronectin polypeptide fragment of any one of Embodiments 1 to 12, further comprising a C-terminal His-tag.

Embodiment 14. The vitronectin polypeptide fragment of Embodiment 13, comprising one, two, three, four, five, six, seven, eight, or more C-terminal His residues.

Embodiment 15. A vitronectin polypeptide fragment consisting of SEQ ID NO: 3.

Embodiment 16. The vitronectin polypeptide fragment of any one of Embodiments 1 to 15, wherein the vitronectin polypeptide fragment is animal-free.

Embodiment 17. The vitronectin polypeptide fragment of any one of Embodiments 1 to 16 conjugated to a microcarrier.

Embodiment 18. The vitronectin polypeptide fragment conjugate of Embodiment 17, wherein the microcarrier comprises a hydrogel or polystyrene microsphere.

Embodiment 19. A composition comprising the vitronectin polypeptide fragment of any one of Embodiments 1 to 16 or the vitronectin polypeptide fragment conjugate of Embodiment 17 or 18.

Embodiment 20. The composition of Embodiment 19, wherein the composition is animal-free.

Embodiment 21. The vitronectin polypeptide fragment of any one of Embodiments 1 to 16, the vitronectin polypeptide fragment conjugate of Embodiment 17 or 18, or the composition of Embodiment 19 or 20 for use as a cell culture substrate.

Embodiment 22. A nucleotide sequence encoding the vitronectin polypeptide fragment of any one of Embodiments 1 to 16.

Embodiment 23. An expression vector comprising the nucleotide sequence of Embodiment 22.

Embodiment 24. The expression vector of Embodiment 23, wherein the expression vector comprises an E. coli. expression vector.

Embodiment 25. A host cell comprising the nucleotide sequence of Embodiment 22 or the expression vector of any one of Embodiments 23 or 24.

Embodiment 26. The host cell of Embodiment 25, wherein the host cell comprises E. coli.

Embodiment 27. A method of producing a vitronectin polypeptide fragment, the method comprising expressing the vitronectin polypeptide fragment from the nucleotide sequence of Embodiment 22, the expression vector of Embodiment 23 or 24, or the host cell of Embodiment 25 or 26.

Embodiment 28. The method of Embodiment 27, wherein the vitronectin fragment polypeptide produced is animal-free.

Embodiment 29. A cell culture method, the method comprising culturing cells on a substrate comprising the vitronectin polypeptide fragment of any one of Embodiments 1 to 16, the vitronectin polypeptide fragment conjugate of Embodiment 17 or 18, or the composition of Embodiment 19 or 20.

Embodiment 30. The cell culture method of Embodiment 29, the cells comprising stem cells.

Embodiment 31. The cell culture method of Embodiment 30, the stem cells comprising human induced pluripotent stem cells (iPSCs).

Embodiment 32. The cell culture method of Embodiment 30, the stem cells comprising human mesenchymal stem cells (MSCs).

Embodiment 33. The cell culture method of any one of Embodiments 29 to 32, wherein the cells are cultured under animal-free conditions.

Embodiment 34. The cell culture method of any one of Embodiments 29 to 33, wherein cell culture is ex vivo.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Vitronectin 62-292 and VTN62-292,D80Y

The production of a full length recombinant vitronectin (VTN) under animal-free conditions is problematic, resulting in insoluble protein and low yields when expressed in an E. coli protein expression system (Table 1). With this example, various vitronectin variants with improved solubility and expression were produced. The secondary structure of the vitronectin polypeptide and the variants produced and tested in this example are illustrated in FIG. 1.

TABLE 1
Human Vitronectin Manufacturability

| | | Estimated | Estimated Yield | Comments on purification |
|---|---|---|---|---|
| No. | hVTN Construct | Solubility (%) | (tagged): mg/L | from E. coli |
| 1 | 20-478, His | 0% | Low (<1 mg/L) | Insoluble with extremely low |
| | | | | expression levels. |
| 2 | 62-478, His | <5% | Low (<2 mg/ml) | Mostly insoluble with low |
| | | | | expression levels |
| 3 | 62-398, His | 80% | Low (<1.5 mg/L) | Soluble but low expression levels |
| 4 | 62-155, His | 98% | 85 | Soluble with moderate expression |
| 5 | 62-120, His | 98% | 90 | Soluble with moderate expression |
| 6 | 62-292, His | 85-90% | 110 | Soluble with good expression |
| 7 | 62-292, D80Y, His | 95% | 104 | Soluble with good expression |
| 8 | 62-292, Q148E, His | 95% | 95 | Soluble with good expression |
| 9 | 62-292, T63G, His | >95% | 110 | Soluble with good expression |
| 10 | 62-262, V67S, His | >90% | >100 mg/L | Soluble with good expression |
| 11 | 62-262, T63G, V67S | >95% | >100 mg/L | Soluble with good expression |
| | His | | | |
| 12 | 62-292, D80Y, Q148E, | 95% | >100 mg/L | Soluble with good expression |
| | His | | | |
| 13 | 62-292, | 95% | >100 mg/L | Soluble with good expression |
| | T63G/V67S/D80Y, | | | |
| | His | | | |
| 14 | 62-292, | 80% | 120 | Soluble with good expression |
| | T63G, V67N, F68P, | | | |
| | His | | | |
| 15 | 62-292, T63G, | 80% | 150 | Soluble with good expression |
| | V67S, F68P, His | | | |

Previous work suggested that removal of the N-Terminal SMB domain alone (VTN-N, or VTN62-478) or in conjunction with the deletion of the C-terminal V10 domain (VTN-NC, or VTN62-398) resulted in a recombinant VTN variant made in E. coli that supported pluripotent stem cells in culture. Although these variants retain or even improve adhesion and survival bioactivity in culture, they result in insoluble protein when made in E. coli, which requires additional solubilization steps complicating large scale manufacturing. The poor solubility and the resulting low yields of these two VTN variants (VTN62-478 and VTN62-398 is shown in Table 1.

The vitronectin variants produced and tested in this example retain the integrin binding RGD site at amino acids 64-66, while the compact N-terminal somatomedin B (SmB) domain (at amino acids 20-63) was deleted. The heparin binding (Hb) region (at amino acids 362-395) was in some of the variants produced and tested in this example. See FIG. 1.

The VTN62-292 variant was constructed to eliminate the disulfide bond between cysteine residues 293 and 430 (see FIG. 1 and Table 1).

With two of the VTN truncation variants produced and tested in this example (VTN62-155 and VTN62-120) glycosylation sites at amino acids 169 and 242 were eliminated (See FIG. 1 and Table 1).

In standard E. coli cultures for the heterologous expression of recombinant protein, the protein yield of these various constructs was tested. While the expression of longer fragments up to amino acid 398 resulted in low solubility and poor yields in E. coli, the VTN62-120, VTN62-155, and VTN62-292 variants were soluble and resulted in high expression (see FIG. 1 and Table 1). Due to the poor yields and/or low solubility, variants 1-3 from Table 1 were incompatible with animal-free production in E. coli and were eliminated from further analysis.

Next, this example identified soluble variants with high yields that are suitable substrates for cell culture. To this end two cell types important for potential cell therapy applications were tested, human induced pluripotent stem cells (iPSCs) and mesenchymal stem cells (MSCs). One important goal of stem cell workflows is to eliminate animal containing raw materials. Therefore, the VTN variants were tested in a unique media formulation wherein the components, including all growth factors, were animal-free.

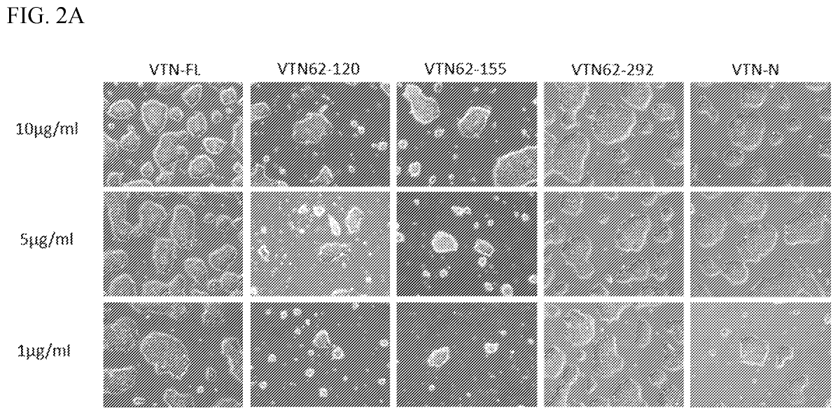

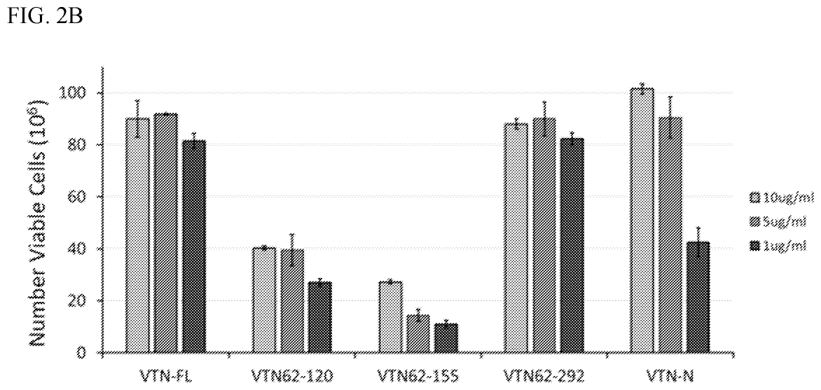

The iPSC line 12-10a was plated as single cells at 20,000 cells/cm² and allowed to grow colonies for 5 days with daily media exchanges. Three different concentrations of a given VTN variant were tested, including more typical concentrations of 10 μg/ml and 5 μg/ml and a low concentration of 1 μg/ml, to discern more subtle performance differences. Indeed, the iPSCs cultured on the VTN62-292 variant displayed typical morphology and high expansion even at low concentrations compared to the full-length animal derived VTN and the commercially available, VTN62-498 variant (VTN-N, commercially available from ThermoFisher) (FIGS. 2A and 2B). However, the VTN62-120 and VTN62-155 variants did not support iPSC colony growth, especially at lower concentrations.

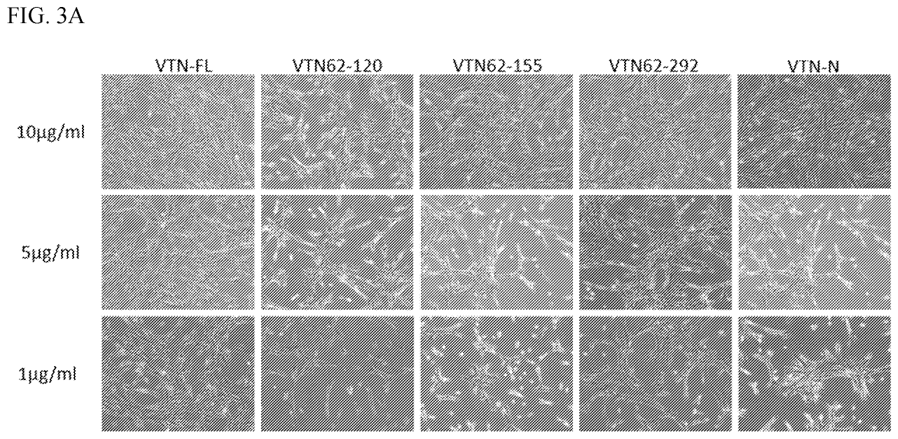

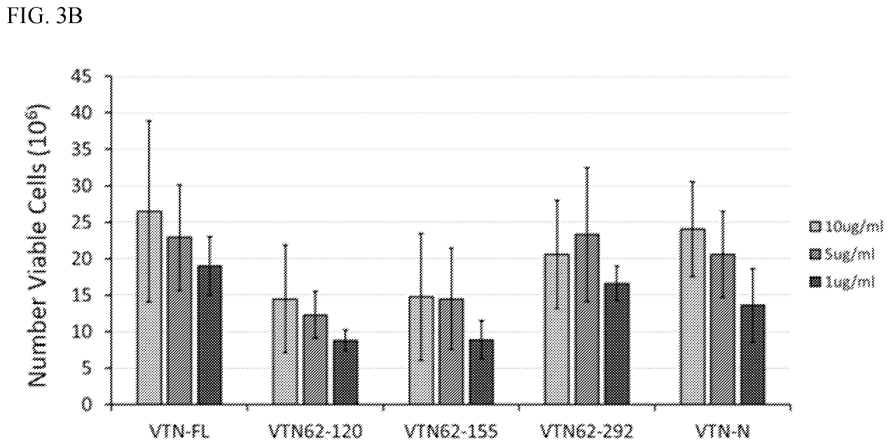

Human MSC cultures should similar, though not as drastic, results (FIGS. 3A and 3B). MSCs did adhere and have normal morphology on VTN62-120 and VTN62-155, but the yield of viable cells was considerably less than on the VTN62-292 variant. These results indicate that the VTN62-292 variant confers similar biological properties to the full-length wildtype VTN in its ability to support stem cell culture.

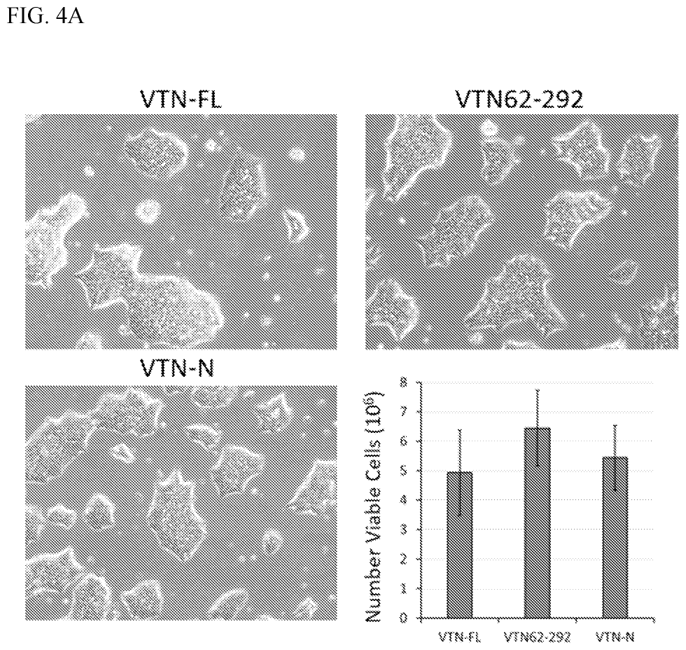

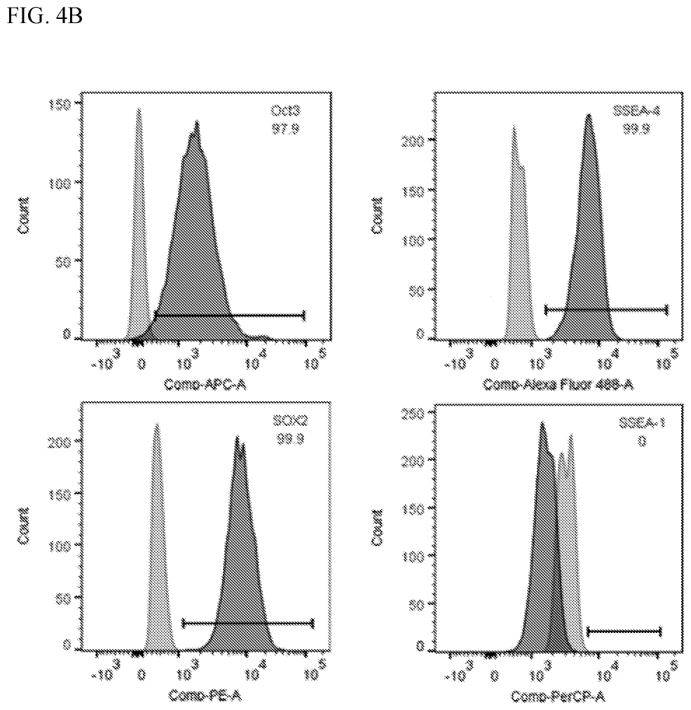

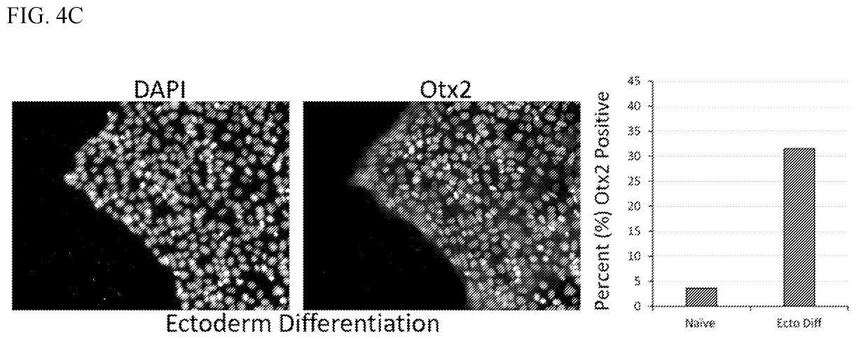

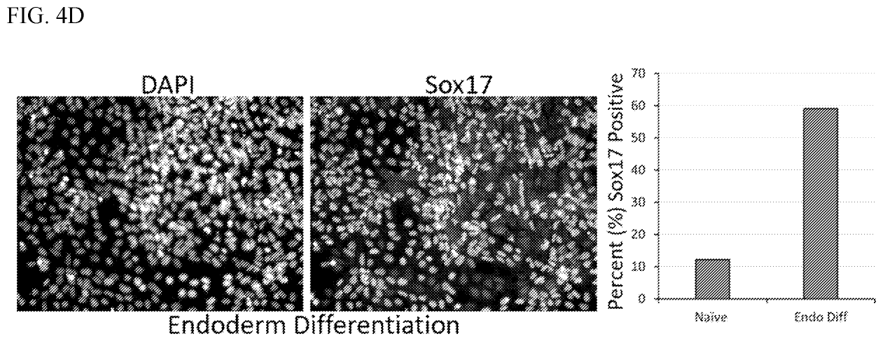

An important feature of pluripotent stem cells is their ability to proliferate while maintaining potency over long periods of time and after multiple passages. Therefore, the ability of the VTN62-292 variant to support the expansion and stemness iPSCs over time was tested. After 5 passages (3 weeks in culture), iPSCs maintained typical morphology with high expansion rates (FIG. 4A). Using flow cytometry, it was shown that the pluripotency markers, Oct3, Sox2, and SSEA-4 remained highly expressed while the differentiation marker SSEA-1 was expressed at low levels in cells grown on VTN62-292 for extended periods in cell culture (FIG. 4B).

Next, it was demonstrated that iPSCs maintained on VTN62-292 could differentiate into different germ layers and diverse lineages. Ectoderm is patterned with an inhibition of TGFβ signals via noggin. Under these conditions, iPSCs lose the expression of Oct3/4 while the ectoderm transcription factor Otx-1 increases significantly (FIG. 4C). Definitive endoderm develops in vivo through combination of Activin A and Wnt signaling. Emulating these signals ex vivo results in increased expression of Sox 17 and reduction of Oct3/4 in iPSCs maintained on VTN62-292, thus showing the potential of these cells to differentiate into endoderm lineages (FIG. 4D). In other experiments, iPSCs maintained on VTN62-292 were able to differentiate into cardiomyocytes, a mesoderm derivative. Thus, iPSCs maintained for long periods of cell culture on VTN62-292 maintain their pluripotency.

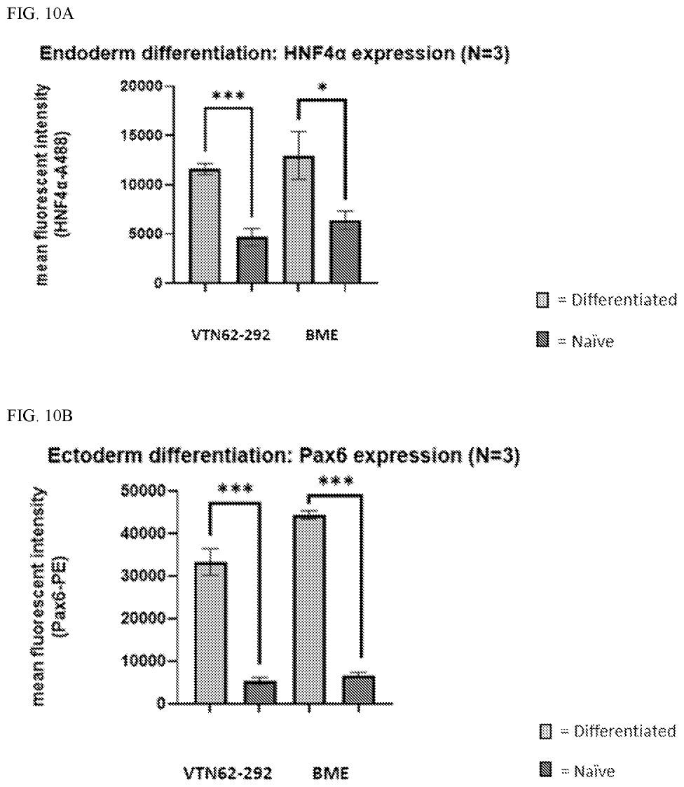

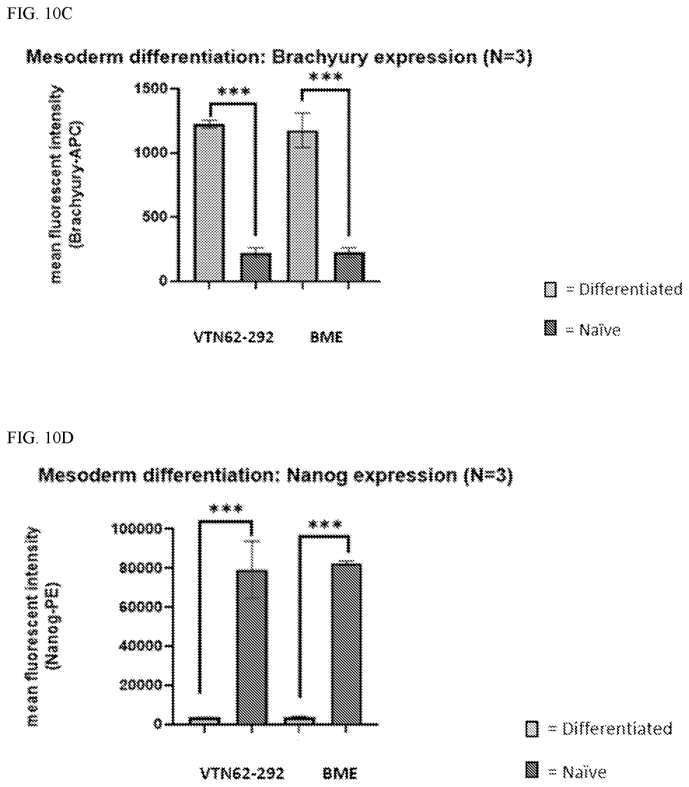

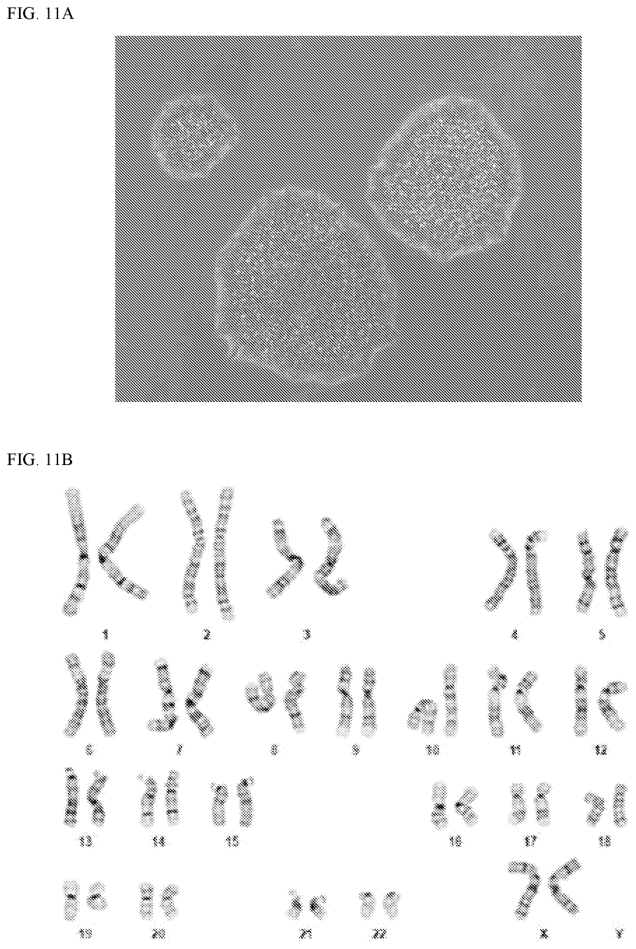

The results shown in FIGS. 4A-4D were confirmed in separate experiments with flow cytometry which showed increased expression Brachyury, HNF4α, and Pax6 for mesoderm, endoderm, and ectoderm, respectively, as shown by the significant increase in mean fluorescent intensity for these markers (FIG. 10A-C). In addition, the stemness marker Nanog significantly decreases following a protocol to induce mesoderm differentiation (FIG. 10D).

In a different cell line, the BXS0114 line (available from ATCC) also displayed pluripotency after multiple passages on VTN62-292 in three experiments. Definitive endoderm differentiation was achieved Wnt3a and Activin signals and assessed with the marker HNF4α by flow cytometry (FIG. 10A). There was significant increase in HNF4α indicating a successful differentiation into endoderm. Differentiation into definitive ectoderm was performed with dual SMAD inhibition with noggin and the small molecule SB413452. Successful differentiation was observed by staining with the neuroectoderm marker Pax6 which increased dramatically following the differentiation protocol (FIG. 10B). Differentiation into Mesoderm lineage was achieved via the small molecule CHIR 99021 a GSK 3 inhibitor. Flow cytometry of brachyury showed a significant increase in mesoderm differentiation (FIG. 10C) and a concurrent decrease in the stemness marker, Nanog (FIG. 10D). Taken together these data show that the VTN62-292 variant support the culture of Pluripotent stem cells under naïve conditions and maintain their capacity for differentiation into all three of the major germ layers.

A potential concern regarding the cultivation and expansion of rapidly dividing stem cells requires attention to the stability of the genome. Therefore, after over 2 months in cell culture and 13 passages, karyotype analysis was performed (WiCell, Madison, Wi). The analysis of 1210a cells indicated a normal karyotype/banding pattern of the chromosomes, which provides evidence for genomic stability (FIGS. 11A and 11B). After long-term culture of iPSCs on VTN62-292 for over 2 months and 13 passages, cells maintain a normal karyotype, indicating genomic stability, an important feature for the propagation of stem cells for regenerative medicine.

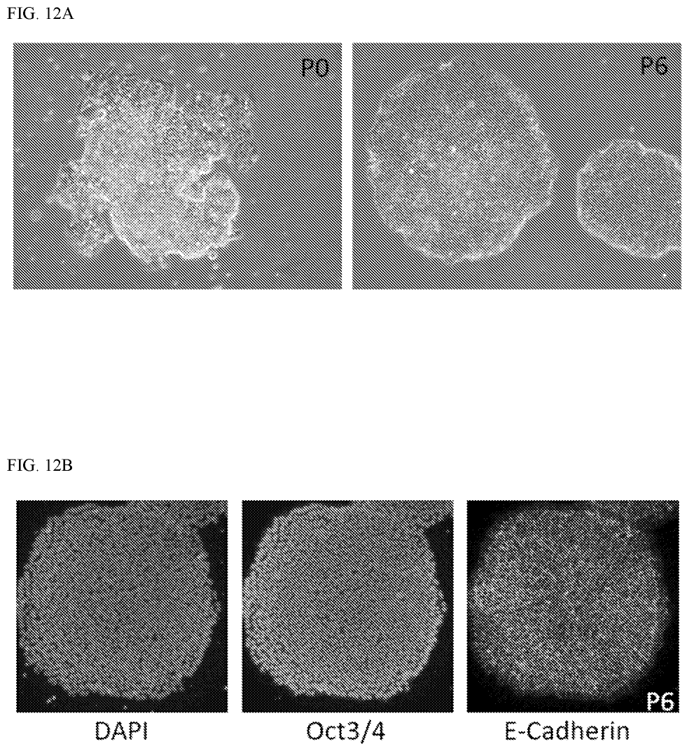

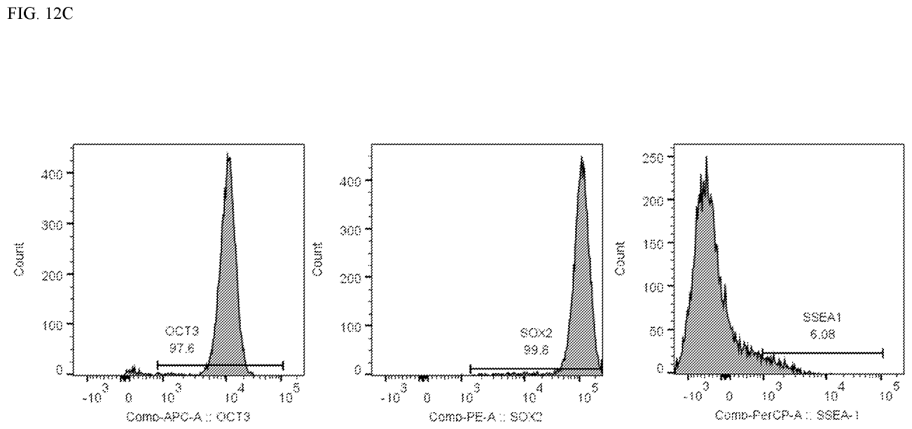

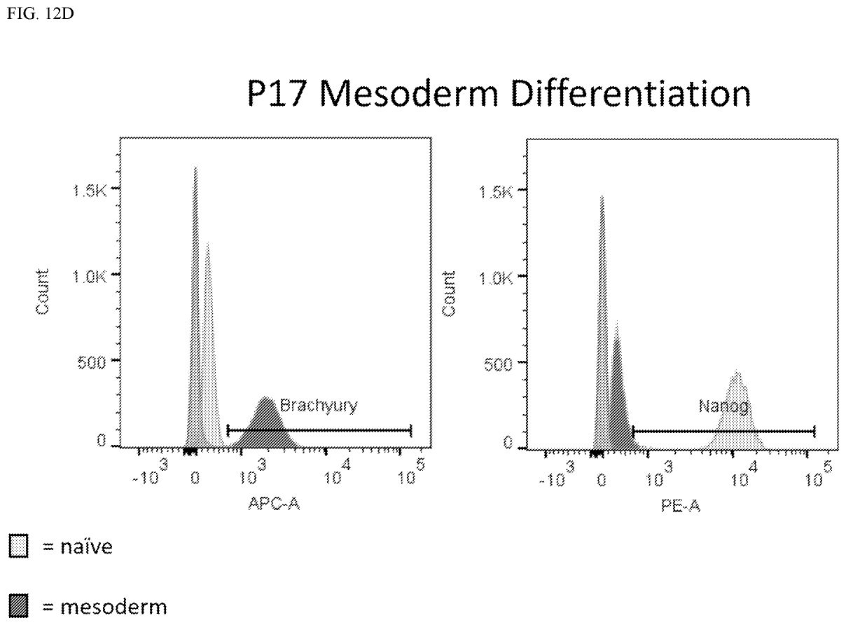

It has been reported that VTN does not support the reprogramming of somatic cells into induced pluripotent stem cells but other undefined matrices like basement membrane extract (BME, aka Matrigel) and laminin can support reprogramming. Since these matrices are not animal free, VTN62-292 was used as a substrate to support an animal free workflow following reprogramming with Yamanaka factors (Oct3/4, Klf4, Sox2, c-Myc), peripheral blood monocytes (PBMCs) were re-programmed into induced pluripotent stem cells with the Sendai reprogramming kit (CytoTune 2.0) using hVTN62-292 as the substrate following a modification of the manufacturer's guidelines. After day 7 of transduction, the culture was fully transitioned into fully animal free conditions. FIGS. 12A-12D show the reprogramming of peripheral blood monocytes (PBMCs) into induced pluripotent stem cells using hVTN62-292 as the substrate. This is significant in that previous work failed to show this with other VTN variants. FIG. 12A shows the formation of iPSC colonies at different times following transduction with the Sendai Cytotune 2.0 reprogramming kit (Thermo-Fischer). After cloning individual colonies, nascent iPSCs derived on hVTN62-292 exhibit the morphological features of iPSCs with tightly packed cells in colonies with smooth borders. These iPSCs display the characteristic markers for iPSCs including Nanog, Oct3/4 and E-Cadherin expression (FIG. 12B). The analysis of Oct3 and Sox2 by flow cytometry also showed the expression of stemness markers while the absence of SSEA1 expression indicating an undifferentiated state of the iPSCs (FIG. 12C). In addition, the iPSCs derived from a single clone have the differentiation capacity to form mesoderm cells as indicated by the increased expression of Brachyury (FIG. 12D).

FIG. 12A shows the formation of iPSC colonies at different times following transduction with the Sendai viruses. The colony formation efficiency was similar to that on a BME matrix. After cloning individual colonies, nascent iPSCs derived on hVTN62-292 exhibit the morphological features of iPSCs with tightly packed cells in colonies with smooth borders. These iPSCs display the characteristic markers for iPSCs including Nanog, Oct3/4 and E-Cadherin expression (FIG. 12B). The analysis of Oct3 and Sox2 by flow cytometry also showed the expression of stemness markers while the absence of SSEA1 expression indicating an undifferentiated state of the iPSCs (FIG. 12C). In addition, the iPSCs derived from a single clone have the differentiation capacity to form mesoderm cells as indicated by the increased expression of Brachyury (FIG. 12D). The reprogramming experiments were successfully repeated in 2 independent experiments with 2 different donors.

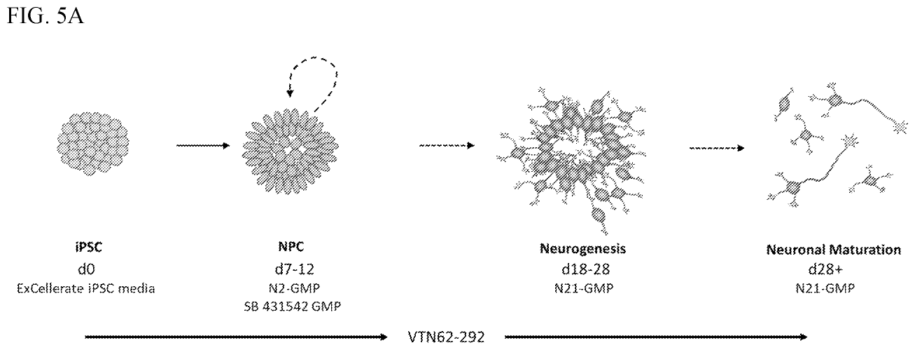

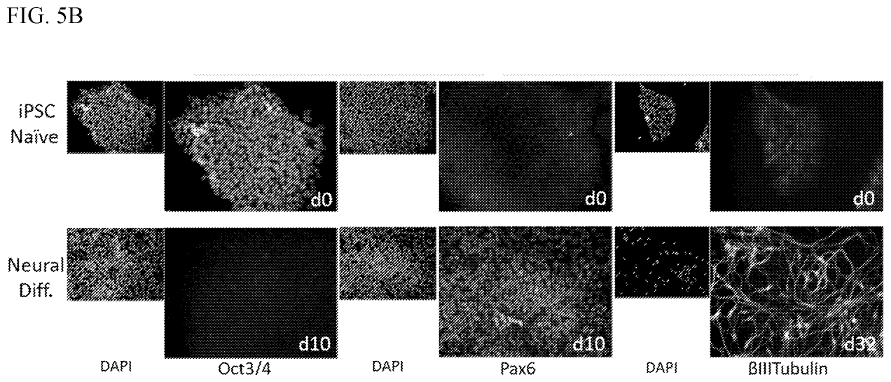

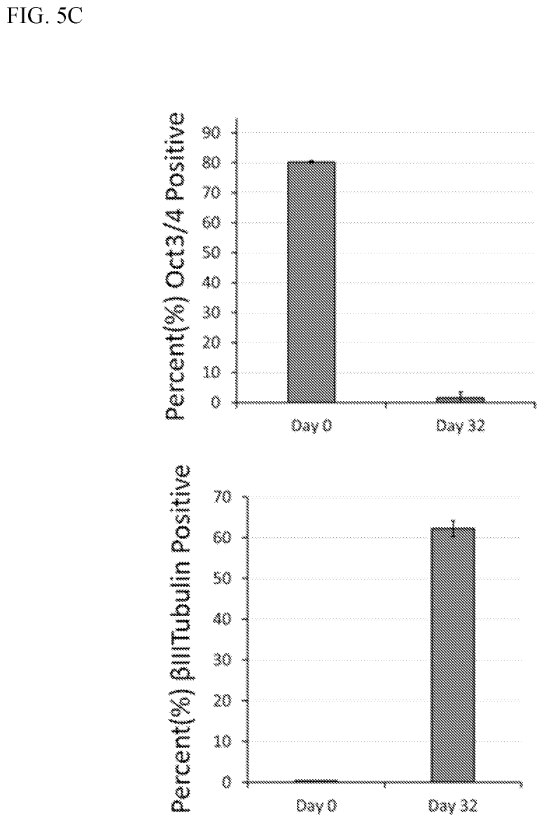

A major goal of this example was to develop a VTN variant for use in a completely animal-free workflow for generating differentiated cells from pluripotent stem cells. Such a variant would have broad application for improving the safety and efficacy of workflows related to regenerative medicine. Thus, VTN62-292 was tested as the cell matrix in a protocol to generate forebrain neurons from iPSCs ex vivo using (FIG. 5A). All other components, including the culture media, supplements, growth factors and small molecules were completely devoid on animal containing materials. After expanding iPSCs for 4 passages on VTN62-292, cells were plated at high density in animal-free N2 media with the TGFβ inhibitor SB43152-GMP (Tocris biosciences) based on previous work (Chambers et al., 2009, Nat Biotechnol; 27(3):275-80; and Chambers et al., 2009, Erratum in: Nat Biotechnol; 27(5):485). After 7-12 days, this results in neural progenitor cells (NPCs) that have high expression of the neural stem cell transcription factors Pax6 and Sox1 (FIG. 5B). These NPCs can then be differentiated further into forebrain neurons after transitioning the cells into a neuronal media with an animal-free N21 neural supplement. After 32 days after the initiation of the differentiation, over 60% of the cells had long neurite extensions and were positive for the neuronal marker beta III tubulin (FIG. 5C). In addition, the expression of the stemness marker Oct3 which was prominent in the iPSCs was absent after the differentiation protocol (FIG. 5C). Taken together, these data indicate VTN62-292 can support iPSCs in the naïve pluripotent state and can also support the differentiation of iPSCs into downstream lineages, indicating that VTN62-292 is a promising agent for stem cell culture.

To improve the function of VTN as an adhesion protein, targeted mutations were introduced in the VTN 62-292 variant.

First, amino acids flanking the RGD integrin binding site were modified. Six mutants in the VTN62-292 variant were produced and tested to determine if these amino acid changes could facilitate improved biological function in the context of the larger VTN polypeptide in cell culture. These are variants 9-15 listed in Table 1. Vitronectin binds integrin heterodimers via the RGD motif. While αVβ5 and αVβ3 are the major VTN receptors binding with high affinity, α5β1 integrins can also interact weakly to VTN. The RGD motif is conserved among other integrin-binding proteins such as fibronectin, laminin and osteopontin, but these proteins show different affinities to integrin heterodimers. The RGD motif itself is critical for the optimal interactions with integrins for any of these proteins; point mutations here decrease or eliminate integrin binding (Cherny et al., 1993, J Biol Chem; 268(13):9725-9). There is mounting evidence that the amino acids sequences flanking the RGD motif can at least partly explain the differential binding of the various proteins binding integrins via this tripeptide. For example, biochemical studies using small peptides have studied the binding affinities to different integrin heterodimers (Kapp et al., 2017, Sci Rep; 7:39805). Equipped with this information, we attempted to modulate VTN62-292 function as an adhesion protein for stem cell culture by modifying the amino acids in the region around the RGD site. For example, the sequence GRGDSP was shown to increase the binding affinity to αVβ5, αVβ3, and α5β1. This is a vast improvement over the RGD peptide in isolation (Kapp et al., 2017, Sci Rep; 7:39805).

Second, inter-species sequence alignments were used to identify consensus sequences in the VTN polypeptide that were different in the human sequence (Jones et al., 2020, Methods Enzymol; 643:129-148). Consensus sequence alignments have been used to study the molecular evolution of proteins and more recently has been used to identify possible function changing mutations. In particular, consensus sequences may indicate differences in stability of the molecular structure, which would be beneficial for recombinant protein expression. Using a publicly available online sequence alignment tool, various single amino acid variations were identified in the human sequence compared to majority of other vitronectin sequences (Jones et al., 2020, Methods Enzymol; 643:129-148). The two most prevalent amino acids changes, an aspartic acid to tyrosine change at position 20 (D80Y) and a glutamine to glutamic acid change at position 148 (Q148E) were further tested. These point mutations were produced and tested to see if they conferred any advantage to the function and stability of the recombinant vitronectin protein in the 62-292 variant. These are the variants 7-8 listed in Table 1.

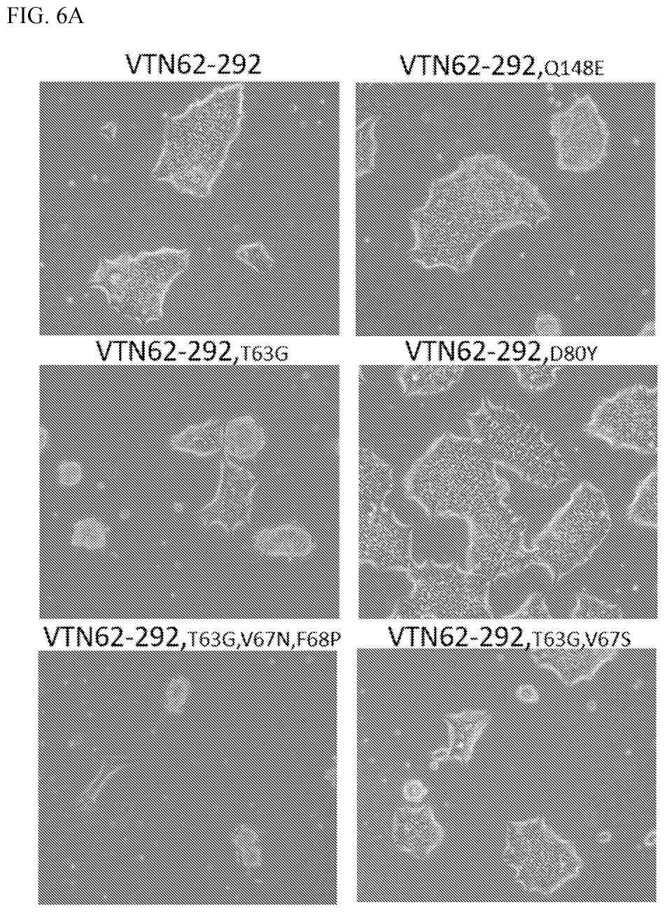

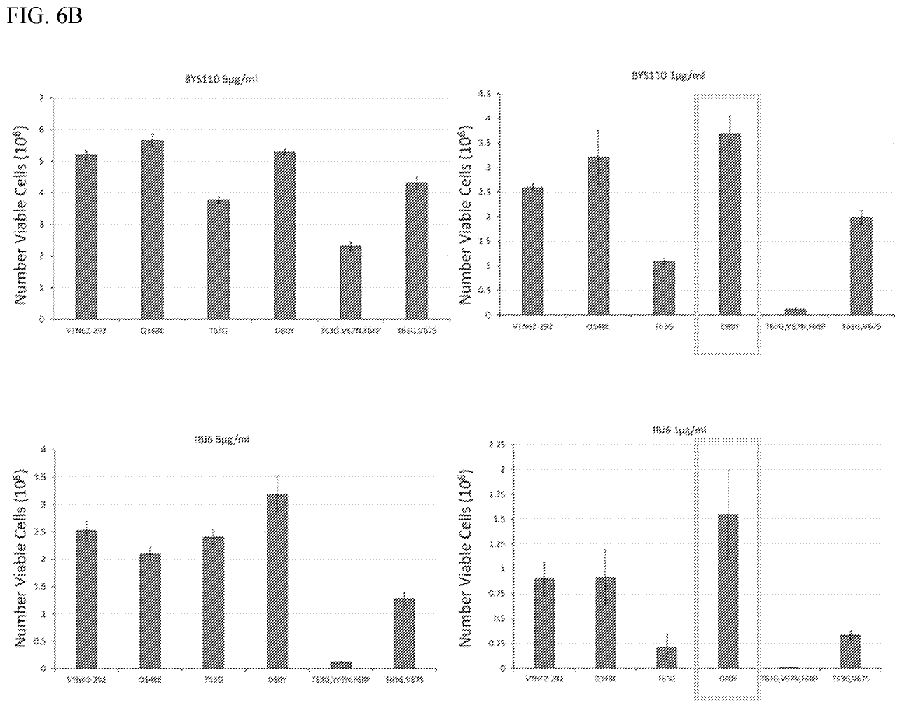

The screening of these mutations to VTN62-292 focused on the ability to support pluripotent stem cell culture. Two different iPSC cell lines (BYS110 and iBJ6) and two concentrations were tested. Surprisingly, the amino acid substitutions predicted to improve integrin binding actually reduced iPSC cell growth (FIGS. 6A and 6B). For example, the triple mutation VTN62-292, T63G, V67N,F68P was expected to improve cell adhesion since peptide studies showed great improvements to the binding of various integrin hetereodimers including αVβ5, αVβ3, and α5β1 (Kapp et al., 2017, Sci Rep; 7:39805). However, in these studies, the mutation was detrimental to the function of vitronectin as a substrate for iPSCs especially at the low concentration (FIGS. 6A and 6B). Interestingly, the two mutations tested using consensus sequence alignment showed promise as a matrix for iPSCs (VTN62-292,Q148E and VTN62-292,D80Y). In particular, the D80Y mutation supported improved cell expansion of both iPSC lines at both concentrations, but more notably at the 1 μg/ml concentration. For the iBJ6 cell line this was an increase of over 50% in total viable cells (FIG. 6B).

In view of the improved cell expansion observed with the VTN62-292 variant and the D80Y mutation, the ability of the VTN variants to bind αVβ5 heterodimers in ELISA assays was tested.

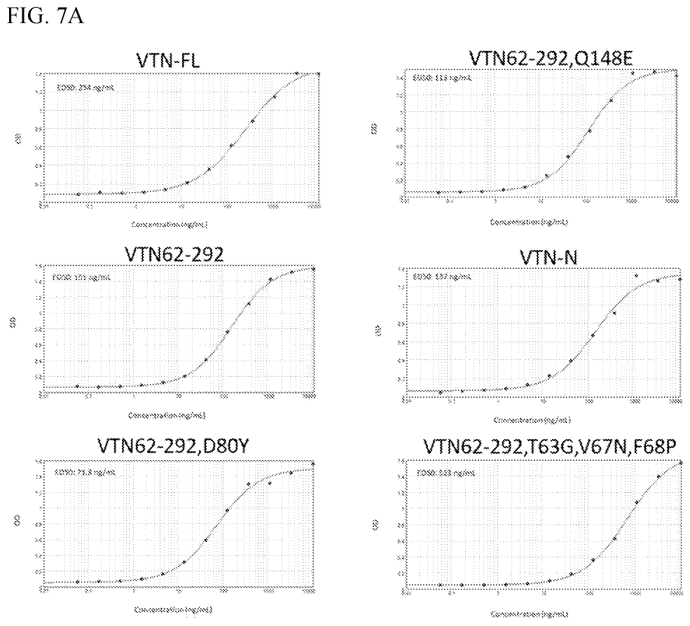

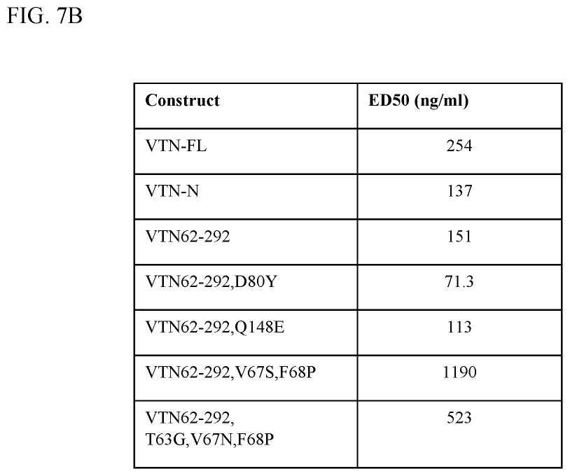

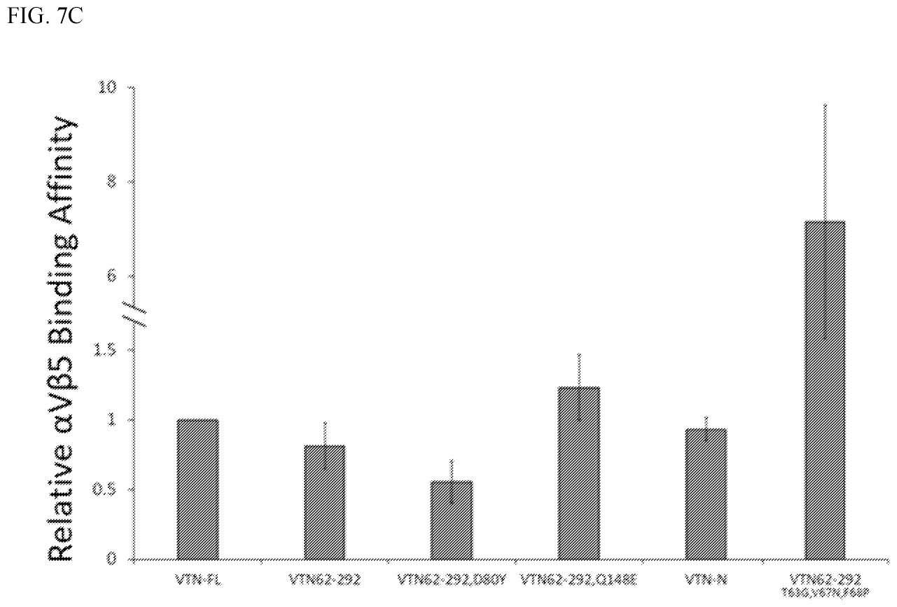

The VTN62-292 variant and mutations therein were compared to full length vitronectin, made in NSO cells (a mouse cell line) and to the commercially available version of VTN62-498 (so called VTN-N, or VTN-CTS, from Thermo-Fisher). These assays demonstrated that VTN62-292 has a similar binding affinity to αVβ5 as full length VTN and VTN-CTS (FIG. 7A) and that the VTN62-292 triple mutant had greatly reduced binding to αVβ5, with an ED50 of 523 ng/ml, nearly a stark increase compared to VTN-FL or VTN62-292 (which indicates poor binding). This nicely correlated with the previous experiments whereby VTN62-292, T63G, V67N,F68P could not support iPSCs in cell culture (FIGS. 7A, 7B, and 7C).

In contrast, the VTN62-292, D80Y mutant showed nearly a 2-fold increase in αVβ5 binding affinity, which correlated to the improved function of this variant in iPSC cell expansion. This is shown by the nearly 50% decrease in the ED50 value for αVβ5 binding (FIG. 7B). The Q148E mutant showed no significant increase in αVβ5 binding. This data indicates that the improved function of the D80Y mutant in supporting stem cell expansion in culture may be related to its increased affinity for integrins and integrin-mediated adhesion or cell signaling.

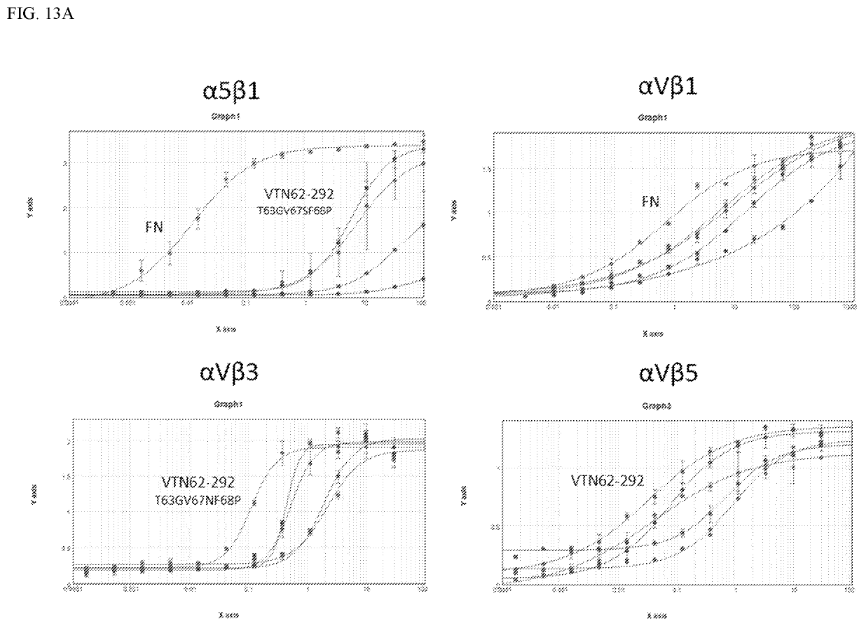

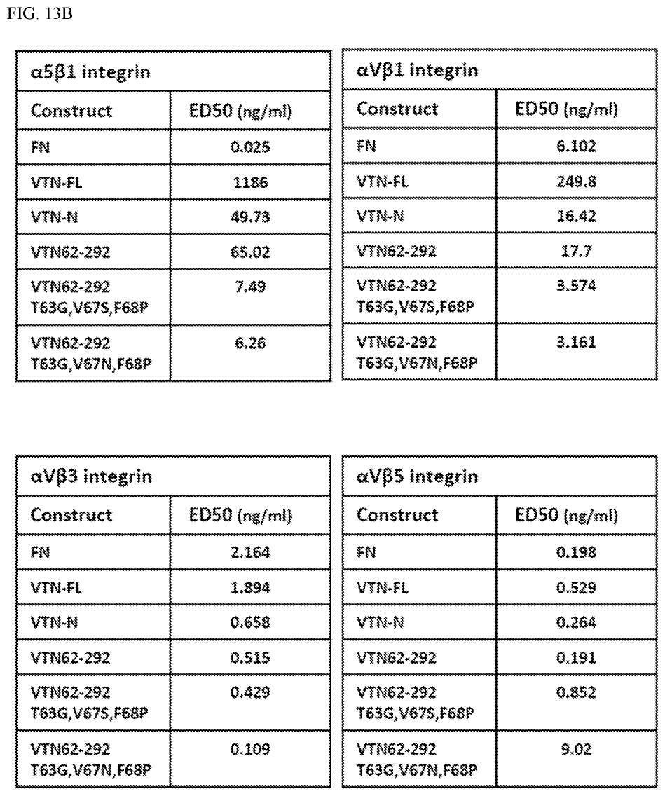

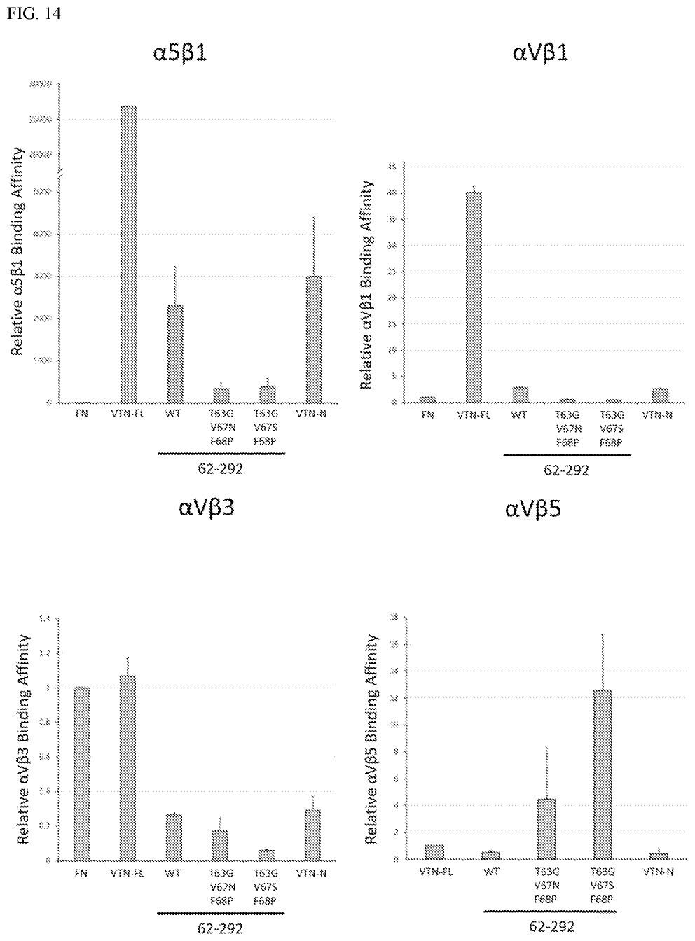

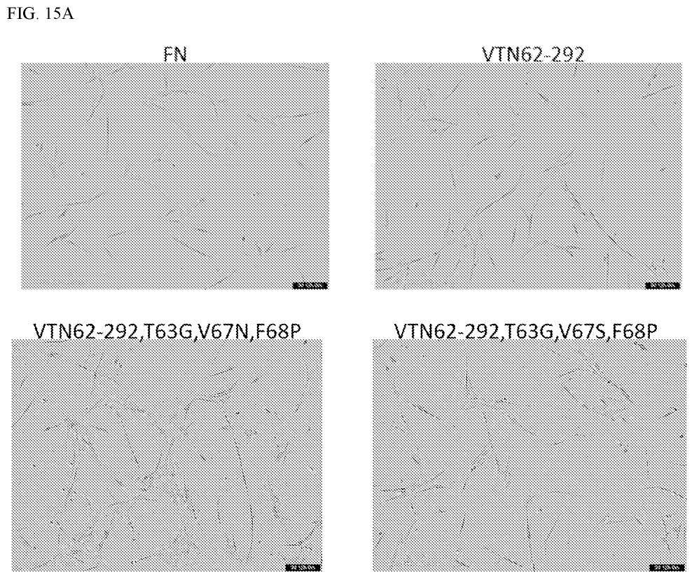

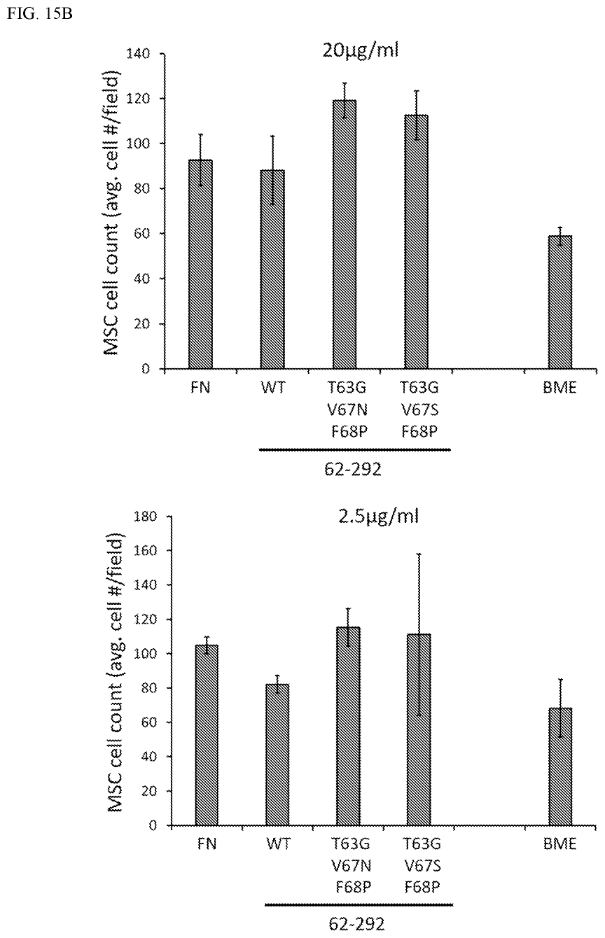

Although the mutants flanking the RGD domain did not show an improvement in αVβ5 binding, there is a possibility that these changes could affect the binding to other integrin heterodimers. Additional ELISA experiments were performed examining the binding affinity of the VTN62-292 variant and mutants made therefrom to the major RGD receptors: alphaVBeta5 (αVβ5), alphaVBeta1 (αVβ1), alphaVBeta3 (αVβ3) and alpha5Beta5 (α5β1). αVβ3 and α5β1 are the major fibronectin (FN) receptors. αVβ3 and αVβ5 are VTN receptors. FN and full length VTN were used for comparisons. FIGS. 13A and 13B and FIG. 14 show the binding affinity of the VTN62-292 variant and mutations flanking the RGD motif to the major RGD-binding integrins heterodimers including alpha VBeta5 (αVβ5), alphaVBeta1 (αVβ1), alphaVBeta3 (αVβ3) and alpha5Beta5 (α5β1). In these assays, hVTN62-292 displays a modest increase, though similar binding affinity to αVβ5 as full-length VTN (VTN-FL). However, hVTN62-292 shows improved binding affinity to αVβ3, αVβ1, and α5β1. This may explain why hVTN62-292 is superior to FL-VTN is some cell growth assays. The VTN-N variant (VTN-CTS) shows a similar trend, but typically with lower affinity for all 4 integrin heterodimers.

These experiments confirmed that the VTN mutants postulated to improve integrin binding, VTN62-292, T63G, V67N, F68P greatly diminished αVβ5 binding affinity, with an increase in the ED50 value. The VTN62-292 T63G, V67S, F68P mutant also showed reduced aVB5 binding affinity, albeit to a lesser degree. Interestingly, the triple mutants did show increased affinity to the two major fibronectin receptors, αVβ3 and α5β1, as well as αVβ1. This is important in that here are mutants that are similar to fibronectin in their integrin binding capacity. Since the VTN62-292 T63G, V67S, F68P and VTN62-292 T63G, V67N, F68P mutants can be made efficiently in animal free conditions while full length fibronectin cannot, these mutants may confer an advantage to achieve animal free cell culture conditions with cell types that utilize FN and/or these integrins for optimized growth.

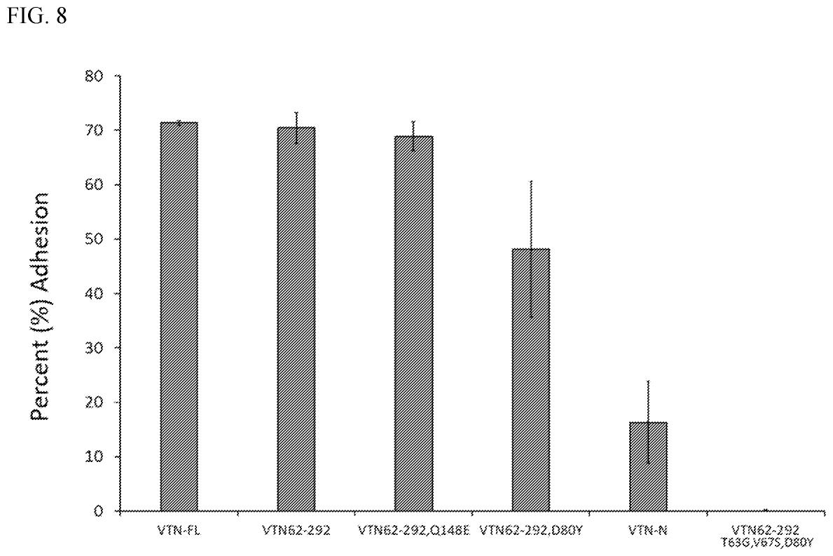

To further investigate the role of VTN variants in supporting iPSCs, a modified adhesion assay was performed whereby cells were allowed to adhere for 1 hour to VTN-coated tissue culture plates before washing and testing for adhesion of cells to the substrate (Kueng et al., 1989, Anal Biochem; 182(1):16-9; and Taooka et al., 1999, J Cell Biol; 145(2):413-20). This assay is considered to be a measure of how tightly cells are bound to the underlying matrix. Using this approach, an increase in iPSC cell binding to VTN62-292 compared to full length VTN (FIG. 7B) was not detected. The VTN62-292 variant did show higher adhesion that the VTN-N (CTS) variant. This is important because the VTN-N is the only other functional animal-free VTN available. However, the D80Y mutation did not result in any improvement in adhesion which was surprising given the improved integrin binding (see FIG. 8). These results may not be fully reflective of the cell adhesion properties of VTN as this assay probes for the nascent adhesions formed during early cell attachment but not more mature focal adhesions.

While the VTN62-292 and VTN62-292, D80Y variants supported iPSC expansion in cell culture, the triple mutants VTN62-292 T63G, V67S, F68P and VTN62-292 T63G, V67N, F68P did not. This is likely a reflection of the diminished capacity for these mutants to bind αVβ5, the major VTN receptor expressed in iPSCs. In data not shown, this example confirmed that iPSCs lack the expression of the typical VTN receptor β3 but mesenchymal stem cells (MSCs) express all the major subunits for RGD receptors including β3. MSCs are commonly grown on FN and MSC differentiation is facilitated by α5β1 integrins. Therefore, it is possible that the VTN62-292 T63G, V67S, F68P and/or the VTN62-292 T63G, V67N, F68P mutants could facilitate the growth of MSCs given the ELISA data showing the increased affinity for αVβ3 and α5β1.

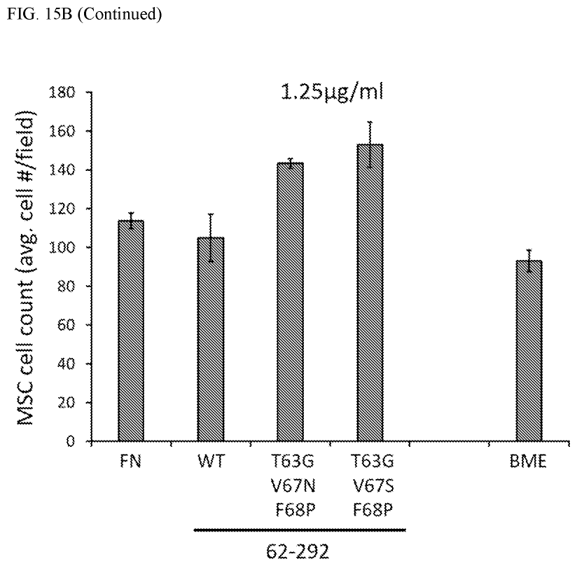

To test this, human bone derived MSCs were cultured on FN, VTN62-292, VTN62-292 T63G, V67S, F68P and VTN62-292 T63G, V67N, F68P (FIGS. 15A and 15B). At various concentrations VTN62-292 T63G, V67S, F68P and VTN62-292 T63G, V67N, F68P showed improved cell growth compared to FN and VTN62-292 (FIG. 15B). All of these matrices had improved MSC growth compared to BME.

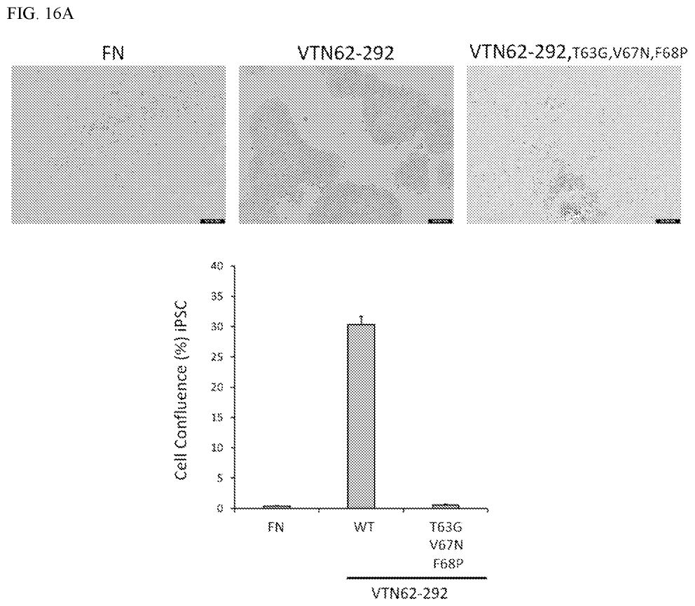

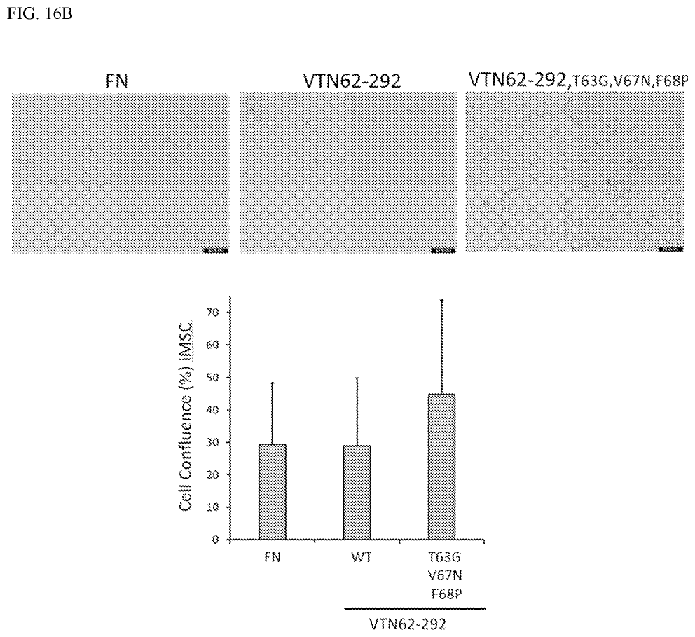

FIGS. 15A and 15B show the effects of VTN mutants on MSC growth in culture. The data presented is from triplicate experiments of three concentrations of VTN. The cell growth was monitored by analyzing the confluence of the MCS cell culture after 72 hours. FIG. 15A shows brightfield images of MSCs on FN and the different VTN62-292 mutants. All three concentrations show that the VTN62-292, T63G, V67N,F68P and VTN62-292, T63G, V67S,F68P mutants support enhanced MSC growth (FIG. 15B). This is in sharp contrast to the growth of iPSCs on the triple mutants which is attenuated (VTN62-292, T63G, V67S,F68P) or completely abrogated (VTN62-292,T63G,V67N,F68P) (FIGS. 6A and 6B). FIGS. 16A and 16B show the cell expansion of the IBJ6 iPSC cell line in the undifferentiated state and following differentiation into MSCs on FN, VTN62-292, and VTN62-292, T63G, V67N,F68P. This experiment shows that the same cell line at different states of stemness can switch their substratum preference. In the undifferentiated state, iBJ6 iPSCs only expand significantly on VTN62-292 as seen in brightfield images and quantified by percent confluence (FIG. 16A). Note there is essentially no growth on VTN62-292, T63G, V67N,F68P. Following differentiation to induced MSCs (iMSCs), the same cell line show increased growth on FN but the best growth on VTN62-292, T63G,V67N,F68P (FIG. 16B). This is likely due to the increased expression of αVβ3 following MSC differentiation and/or increased binding through α5β1 integrins.

MSCS derived from iPSCs (iMSCs) offer many advantages for clinical applications including homogeneity and scalability. Therefore, the VTN62-292 and VTN62-292 T63G, V67N, F68P were tested for suitability for iMSCs. First iBJ6 iPSCs were grown on FN, VTN62-292 and VTN62-292 T63G, V67S, F68P. Brightfield images and cell confluence measurements confirmed that VTN62-292 supported robust expansion of iPSCs while FN and VTN62-292 T63G, V67S, F68P failed to support iPSC growth (FIG. 16A). However, following iPSC differentiation of into iMSCs, FN, VTN62-292 and VTN62-292 T63G, V67S, F68P supported cell expansion (FIG. 16B). The VTN62-292 T63G, V67S, F68P mutant even showed the highest cell expansion as indicated by cell confluence measurements. This likely reflects the increased expression of β3 following MSC differentiation and/or increased signaling through α5β1.

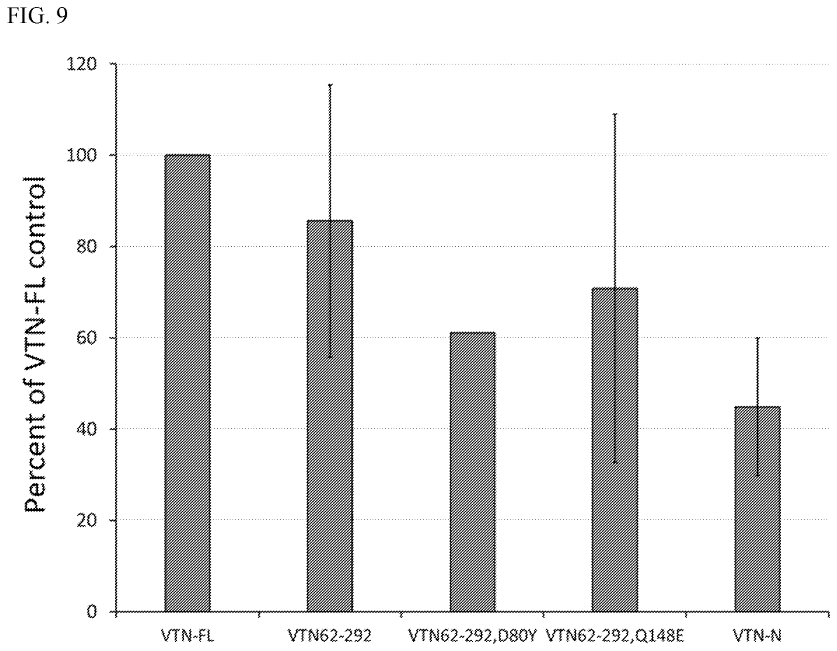

This improved function of VTN62-292 and the D80Y mutant may be related to protein thermostability, which is predicted based on the previous work with consensus alignments in other proteins (Porebski and Buckle, 2016, Protein Eng Des Sel; 29(7):245-51). Thus, a simple test was performed, relevant for the goals of improving VTN as a cell culture substrate. In this test, VTN variants were incubated at 37° C. for 4 days before plating iPSCs. After 4 days of cell expansion, it was unexpectedly observed that the D80Y mutation did not confer any additional stability above the VTN62-292 mutant (FIG. 9). Although moderate, the VTN62-292 variant did confer a stability advantage compared to the VTN-N variant. However, none of the E. coli derived fragments had a stability advantage over the VTN-FL proteins. Nevertheless, these results suggest that VTN62-292 may have increased stability over the other E. coli derived fragments, an additional advantage for cell culture.

Taken together, the results of this example indicate that VTN62-292 and VTN62-292,D80Y variants confer significant advantages over full length VTN and previously published VTN variants. First, the enhanced manufacturability of the VTN62-292 in prokaryotic expression systems is a major advantage, providing for the production of large quantities of a biologically active adhesion molecule for animal-free workflows for stem cell culture and differentiation. Although smaller variants also showed high expression, their poor function supporting iPSC or MSC culture renders them inadequate for stem cell applications. A second advantage is the improved function of these variants compared to full length VTN or the VTN-N variant for supporting the expansion of iPSCs and MSCs. Regarding iPSCs, this is particularly evident at lower concentrations in which VTN62-292 and VTN62-292,D80Y show greatly improved cell expansion in multiple iPSC cell lines. Moreover, the utility of these variant also applies to more robust stem cell workflow, since they also support the differentiation of iPSCs into definitive endoderm, neural progenitor cells and fully differentiated neurons. And a third advantage is the application to completely animal-free ex vivo workflows related to regenerative medicine.

In addition, the results here suggest that RGD integrin binding specificity can be modulated in the context of a VTN variants to tailor cell-type-specific cell culture and even modify the behavior of these cells. In the context of regenerative medicine, these VTN mutants provide an animal-free substate for the enhanced expansion of MSCs. These mutants, including VTN62-292 T63G, V67S, F68P and VTN62-292 T63G, V67N, F68P, could also prove beneficial to cellular process that are enhanced by α5β1 and/or αVβ3, such as mesoderm lineage differentiation, cardiac differentiation, or osteogenesis.

### Example 2

### Recombinant Vitronectin Variants Tuned for Integrin Specificity Differentially Optimize Growth of Mesenchymal Stem Cells and Pluripotent Stem Cells

Vitronectin (VTN) is a key defined substrate for ex vivo expansion of human induced pluripotent stem cells (iPSCs) but used less often for mesenchymal stem cells (MSCs). The latter cell type is more frequently expanded using fibronectin (FN), which is difficult and expensive to produce using recombinant technology.

The targeted mutations described in more detail in Example 1 improve the ability for VTN to support MSCs through the modified engagement of different integrin heterodimers. These novel recombinant human vitronectin (hVTN) variants show differential binding activity to major RGD integrin heterodimers, including the canonical FN receptor, alpha5-beta1. Through this distinct integrin binding activity, these hVTN variants differentially support the adhesion, survival, proliferation, and differentiation of MSCs and iPSCs. While one of these modified VTN variants (VTN62-292 T63G, V67N, F68P; also referred to herein as VN-3) failed to support undifferentiated iPSCs in cell culture, it showed improved MSC adhesion, survival, and expansion. Moreover, MSC osteogenic differentiation was even enhanced on this VTN variant. The VTN62-292 T63G, V67N, F68P variant showed similar binding affinity to alphaV-beta3 but greatly diminished affinity to alpha V-beta5 and increased affinity to alpha5-beta1. Using integrin expression profiling, it was shown that while MSCs express all the major RGD-binding integrin heterodimer subunits, iPSCs lack the expression of beta 3 integrin, limiting the repertoire of heterodimers iPSCs can use to bind RGD-containing ECM proteins. Blocking the binding of the integrin alpha Vbeta5 heterodimer impairs iPSCs ability to bind full length VTN or any of the VTN variants but does not affect MSC binding to VTN. Only blocking alpha Vbeta3, alphaVbeta5 and alpha5beta1 greatly diminishes MSC binding to VTN. This strongly suggests that while undifferentiated iPSCs primarily use alphaV-beta5 to bind VTN, MSCs are more versatile and able to use various integrin heterodimers to bind VTN. Interestingly, when iPSCs are differentiated into MSCs, they initiate the expression of beta 3 integrin and then can effectively bind to the VTN62-292 T63G, V67N, F68P variant, confirming how the changing expression pattern of integrins confers sensitivity to different vitronectin variants.

Engagement of integrin receptors influences various short-term and long-term cellular responses that drive diverse and essential cellular behaviors. Through direct linkages to the actin cytoskeleton and signaling through Rho GTPases, integrins regulate actin dynamics and focal adhesion turnover which in turn effects cell-substrate binding, cell polarity and migration. In addition, integrins signal through diverse interconnected transduction pathways impinging on cell survival, proliferation, and differentiation. Integrins are transmembrane heterodimers composed of an α and β subunit which can arrange in different combinations to form 24 unique integrin receptors. These different heterodimers can be classified into subgroups according to their binding of different extracellular matrix (ECM) proteins such as collagen, laminin, fibronectin (FN), and vitronectin (VTN).

Fibronectin and Vitronectin share a common arginine-glycine-aspartate (RGD) motif which is recognized by five αV integrins, two β1 integrins and the αIIbβ3 integrin (Humphries et al., 2006, J Cell Sci; 119(Pt 19):3901-3). Additional ECM and adhesion molecules also have RGD sites recognized by these integrins including laminin, osteopontin, bone sialoprotein, and platelet endothelial cell adhesion molecule. While the mechanism for the RGD binding motif is generally conserved amongst the various α/β dimers, there are different ligand affinities, presumably due to specific conformational differences in the binding sites (Wang et al., 2013, Int J Mol Sci; 14(7):13447-62).

It has been well known that the presentation of the RGD motif in different conformations can change the binding of synthetic peptides, with cyclic RGD peptides showing higher affinity that linear peptides and different variations of cyclic peptides displaying different binding affinities to different integrins (Kapp et al., 2017, Sci Rep; 7:39805). Indeed, integrin specificity can be achieved with synthetic peptides by altering the flanking amino acid sequences and the binding mode of the RGD peptides at the interface of the dimers (Kapp et al., 2017, Sci Rep; 7:39805). These studies suggest that the relative affinities a given RGD-containing ECM molecule has for different integrin dimers is dependent on the presentation of the RGD motif, which can be influenced by the flanking amino acid sequence.

Differences in the binding affinities of the integrins for the various RGD containing molecules likely regulate diverse cellular behaviors on ECM matrices in cell culture. The selection of the ECM substrate becomes particularly critical under serum-free cell culture conditions since the normally high levels of fibronectin, vitronectin and other adhesion proteins in serum are necessary for cell adhesion and a plethora of other growth factors, proteins and other molecules influence cell behavior. Pluripotent stem cells (PSCs) and mesenchymal stem cells (MSCs) are two important cell types that require ex vivo expansion on ECM matrices for applications in regenerative medicine. The adoption of these cell types and their derivatives entails optimizing defined cell culture conditions with the elimination of animal-derived and even human-derived material. Under xenogeneic free conditions, protocols for MSC culture commonly use fibronectin for both the expansion phase and differentiation phases (Cimino et al., 2017, Stem Cells Int; 2017:6597815; and Basoli et al., 2021, Sci Rep; 11(1):13089). While used less often, MSCs also propagate and differentiate on vitronectin, basement membrane extract, and laminin (Lam and Longaker, 2012, J Tissue Eng Regen Med, 6 Suppl 3(0 3):s80-6). The capability for MSCs to use diverse substrates is likely due to the expression of diverse integrin subunits (Frith et al., 2012, Stem Cells Dev; 21(13):2442-56). Although MSCs can bind to and signal through various integrins, the expansion rates and differentiation capacity fluctuate depending on the substrate/binding motif. Many studies use FN under xeno-conditions to promote MSC expansion, osteogenic and chondrogenic differentiations which appears to be mediated through both αV and β1 integrins (Singh and Schwarzbauer, 2012, J Cell Sci; 125(Pt 16):3703-12; Di Benedetto et al., 2015, Stem Cell Res; 15(3):618-628; and Basoli et al., 2021, Sci Rep; 11(1):13089) with evidence of enhanced the engagement of α5β1 playing a role in osteogenic differentiation (Martino et al., 2008, Biomaterials; 30(6):1089-97; Hamidouche et al., 2009, Proc Natl Acad Sci U S A; 106(44):18587-91; Frith et al., 2012, Stem Cells Dev; 21(13):2442-56). Although not as commonly used as a substrate for MSCs, vitronectin also supports the adhesion, expansion, and differentiation of MSCs which express the canonical, αVβ3, and alternative, αVβ5, VTN receptors (Ode et al., 2010, J Biomed Mater Res A; 95(4):1114-24). The transition to culture pluripotent stem cells under xenogeneic free cultures began with the adoption of basement membrane extract (BME) as a substrate and media containing key growth factors in lieu of a mouse fibroblast feeder system (Ludwig et al., 2006, Nat Biotechnol; 24(2):185-7). BME is an undefined mixture of proteins with high levels of laminin, collagen IV, perlecan, and entactin as well as lower levels of many other adhesion related proteins such as fibronectin. In the naïve state, PSCs can be expanded and cultured long-term on purified or recombinant ECM proteins including laminin, vitronectin and fibronectin. However, there are inconsistent reports using fibronectin for maintaining PSCs in a pluripotent state, with some reporting lower growth rates and increased spontaneous differentiation (Hayashi et al., 2016, Stem Cells Int; 2016:5380560).

The specific engagement of different RGD integrin receptor dimers have different consequences on intracellular signaling and cell behaviors. For example, the main FN receptors αVβ3 and α5β1 synergize for proper mechanosensing of the microenvironment and motility (Schiller et al., 2013, Nat Cell Biol; 15(6):625-36) but they transduce signals intracellularly via distinct binding partners and pathways. This can lead to nuanced differences in cell behavior in the short term and more conspicuous long-term changes in cellular physiology. In the short-term, FN engagement of α5β1 results in larger more dynamic cells with small focal adhesions whereas FN signaling through αVβ3 results in smaller less motile cells with large focal adhesions and stress fibers (Schiller et al., 2013, Nat Cell Biol; 15(6):625-36). In the long-term, there is evidence that α5β1-mediated signaling is important for osteogenic differentiation for MSCs (Martino et al., 2008, Biomaterials; 30(6):1089-97; Frith et al., 2012, Stem Cells Dev; 21(13):2442-56) and cardiac specification for iPSCs (Neiman et al., 2019, Sci Rep; 9(1):18077). The α5β1 integrin is a main receptor for fibronectin and binds primarily via the RGD site in 10th FNIII repeat domain of central binding region with some additive effects of the PHSRN motif in FNIII repeat domain 9 (Pankov and Yamada, 2002, J Cell Sci; 115(Pt 20):3861-3). Fibronectin is essential for embryonic development; its deletion results in severe mesodermal defects including vascular and cardiac malformations (George et al., 1993, Development; 119(4):1079-91; and George et al., 1997, Blood; 90(8):3073-81). The genetic ablation of α5 integrin has overlapping mesodermal defects with vascular and cardiac abnormalities, suggesting the FN signaling through α5β1 is at least partly responsible for these development processes (Bouvard et al., 2001, Circ Res; 89(3):211-23; and Liang et al., 2014, Dev Biol; 395(2):232-44).

Fibronectin molecules exist in multiple isoforms that are alternative spliced from the same gene. Generally, FN is a dimer of two large ˜250 kDa glycoprotein subunits that are linked via a disulfide bond (Pankov and Yamada, 2002, J Cell Sci; 115(Pt 20):3861-3). As such, the production of recombinant fibronectin is a painstaking process which results in low yields in mammalian systems and is essentially impossible in E. coli-based expression systems (Staunton et al., 2009, Methods Mol Biol; 522:73-99). Although FN has many integrin binding partners, many of the critical effects of FN are mediated through α5β1. Other RGD proteins such as VTN cannot bind and signal through α5β1 despite basic similarities in the RGD binding pocket (Kapp et al., 2017, Sci Rep; 7:39805).

As discussed in more detail in Example 1, a VTN variant consisting of amino acids 62-292 that can be effectively produced in an animal free system with functional activity matching or improving upon full length VTN or other published fragments. Based on the analysis of flanking amino acid sequences from various RGD containing ECM molecules and on modifications to RGD containing peptides (Kapp et al., 2017, Sci Rep; 7:39805), with this example, the modification of the integrin binding site in this VTN62-292 variant provided a platform to specifically engage different integrin isoforms.

With this example, vitronectin variants that modify the binding to specific integrin heterodimers were made. Single, double, and triple mutations designed around the RGD domain in the VTN62-292 variant background show different affinities for αVβ3, αVβ5, αVβ1, and α5β1 heterodimers in ELISA assays. Surprisingly, it was observed that the single and double mutations abrogate the binding of αVβ3, αVβ1, and α5β1 integrins but triple mutants built upon these single and tiple mutants improved the binding to these same integrins. In cellular assays, improved growth was observed of both bone-derived and iPSC-derived MSCs to these same triple mutants while iPSC growth was attenuated or even completely abolished. The latter result is likely due to the decreased capacity for these triple mutants to engage αVβ5, the main VTN receptor on iPSCs. This was confirmed with antibody blocking experiments with iPSCs cultured on VTN that showed blocking αVβ5 alone greatly diminishes iPSC cell growth and blocking β1 together with αVβ5 completely inhibits iPSC growth. Blocking αVβ3 has no effect on iPSC growth on VTN. MSC derived from iPSCs (iMSCs) growth on VN was not drastically attenuated if αVβ5, αVβ3, and β1 were all blocked simultaneously, which is consistent with the integrin expression profile of these cells. Furthermore, it was observed that osteogenic differentiation was enhanced on the triple mutants of VTN, suggesting that the change in integrin engagement has additional effects on cell physiology. Overall, this example indicates that RGD integrin binding specificity can be modulated in the context of a VTN variant to tailor cell-type-specific cell culture and even modify the behavior of these cells. In the context of regenerative medicine, these VTN mutants provide an animal-free substate for the enhanced expansion of MSCs. These mutants may also prove beneficial to cellular process that are enhanced by α5β1 and/or αVβ3, such as mesoderm lineage differentiations or osteogenesis.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, 10 for variations obvious to one skilled in the art will be included within the invention defined by the claims.

### Sequence Listing Free Text

SEQ ID NO: 1 Full length human vitronectin
SEQ ID NO: 2 Vitronectin polypeptide fragment amino acids 62 to 292 (hVTN (62-292))
SEQ ID NO: 3 Vitronectin polypeptide fragment amino acids 62 to 292 with His tag

1. A vitronectin polypeptide fragment, wherein the vitronectin polypeptide fragment:
comprises the deletion of at least 50 consecutive N-terminal amino acids relative to a full length vitronectin polypeptide having SEQ ID NO: 1;
comprises the deletion of at least C-terminal amino acids 362 to 478 relative to the full length vitronectin polypeptide having SEQ ID NO: 1;
comprises the arginine-glycine-aspartic acid (RGD) integrin binding domain of vitronectin;
does not comprise the N-terminal somatomedin B (SmB) domain of vitronectin;
does not comprise the C-terminal heparin binding domain and the Hp4 domain of vitronectin;
comprises about 95% sequence identity to the same fragment of the full length vitronectin polypeptide having SEQ ID NO: 1; and
wherein the vitronectin polypeptide fragment has a length of about 200 to about 350 amino acids.

2. The vitronectin polypeptide fragment of claim 1, wherein the vitronectin polypeptide fragment comprises the Hp1, Hp2, and/or Hp3 domains of vitronectin.

3. (canceled)

4. The vitronectin polypeptide fragment of claim 1, wherein the vitronectin polypeptide fragment comprises:
the deletion of 61 consecutive N-terminal amino acids relative to a full length vitronectin polypeptide having SEQ ID NO: 1; and
the deletion of C-terminal amino acids 293 to 478 relative to the full length vitronectin polypeptide having SEQ ID NO: 1.

5. The vitronectin polypeptide fragment of claim 1, wherein the vitronectin polypeptide fragment comprises about 95% sequence identity to residues 62 to 292 of the full length vitronectin polypeptide having SEQ ID NO: 1.

6. The vitronectin polypeptide fragment of claim 1, comprising:
an amino acid substitution at a position equivalent to position 63;
an amino acid substitution at a position equivalent to position 67;
an amino acid substitution at a position equivalent to position 68;
an amino acid substitution at a position equivalent to position 80; and/or
an amino acid substitution at a position equivalent to position 148 of full length human vitronectin having SEQ ID NO: 1.

7. The vitronectin polypeptide fragment of claim 6, wherein the amino acid substitution comprises a T63G substitution, a V67S substitution, a V67N substitution, F68P substitution, a D80Y substitution, and/or a Q148E substitution.

8. The vitronectin polypeptide fragment of claim 1, the vitronectin polypeptide fragment consisting of SEQ ID NO: 2.

9. The vitronectin polypeptide fragment of claim 1, the vitronectin polypeptide fragment consisting of SEQ ID NO: 2, having an amino acid substitution at position D80 and/or an amino acid substitution at position Q148 relative to full length vitronectin having SEQ ID NO: 1.

10. The vitronectin polypeptide fragment of claim 9, wherein the amino acid substitution at position D80 comprises a D80Y substitution and/or the amino acid substitution at position Q148 comprises a Q148E substitution.

11. The vitronectin polypeptide fragment of claim 1, the vitronectin polypeptide fragment consisting of SEQ ID NO: 2, having an amino acid substitution at position at a position equivalent to position 63, an amino acid substitution at a position equivalent to position 67, and/or an amino acid substitution at a position equivalent to position 68 relative to full length vitronectin having SEQ ID NO: 1.

12. The vitronectin polypeptide fragment of claim 11, wherein the amino acid substitution at position T63 comprises a T63G substitution, the amino acid substitution at position V67 comprises a V67S substitution or a V67N substitution, and/or the amino acid substitution at position F68 comprises a F68P substitution.

13. The vitronectin polypeptide fragment of claim 1, further comprising a C-terminal His-tag.

14. (canceled)

15. The vitronectin polypeptide fragment of claim 1, the vitronectin polypeptide fragment consisting of SEQ ID NO: 3.

16. (canceled)

17. The vitronectin polypeptide fragment of claim 1 conjugated to a microcarrier.

18. The vitronectin polypeptide fragment conjugate of claim 17, wherein the microcarrier comprises a hydrogel or polystyrene microsphere.

19. A composition comprising the vitronectin polypeptide fragment of claim 1.

20. The composition of claim 19, wherein the composition is animal-free.

21. (canceled)

22. A nucleotide sequence encoding the vitronectin polypeptide fragment of claim 1.

23-26. (canceled)

27. A method of producing a vitronectin polypeptide fragment, the method comprising expressing the vitronectin polypeptide fragment from the nucleotide sequence of claim 22.

28. (canceled)

29. A cell culture method, the method comprising culturing cells on a substrate comprising the vitronectin polypeptide fragment of claim 1.

30-37. (canceled)
